(19) United States
(12) Patent Application Publication
(10) Pub. No.: US 2005/0085516 A1
(43) Pub. Date: Apr. 21, 2005

(54) 2-W-diaminocarboxylic acid compounds

(76) Inventors: Costin Rentzea, Heidelberg (DE); Ulf Misslitz, Neustadt (DE); Matthias Witschel, Bad Durkheim (DE); Cyrill Zagar, Mannheim (DE); Andreas Landes, Romerberg-Heiligenstein (DE)

Correspondence Address: KEIL & WEINKAUF, 1350 CONNECTICUT AVENUE, N.W., WASHINGTON, DC, 20036, US

(21) Appl. No.: 10/496,657

(22) PCT Filed: Nov. 28, 2002
(86) PCT No.: PCT/EP02/13449

Related U.S. Application Data
(60) Provisional application No. 60/334,112, filed on Nov. 30, 2001.

(30) Foreign Application Priority Data
Nov. 29, 2001 (DE) 10158628.0

(51) Int. Cl. A61K 31/44; A61K 31/40; A61K 31/195

(52) U.S. Cl. 514/357; 514/408; 514/562; 514/563

(57) ABSTRACT

2,ω-Diaminocarboxylic acid compounds of formula (I), where X, X¹, Ar^{ω}, Ar² and Y are as defined in claim 1, and their use as herbicides, are described.

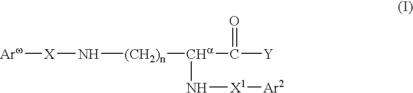

The present invention relates to 2,ω-diaminocarboxylic acid compounds, to their use as herbicides and to their use for the dessication and/or defoliation of plants.

The development of novel herbicides is of particular interest with respect to avoiding the formation of resistance. Many known herbicides furthermore require high application rates, which are uneconomical and ecologically questionable.

It is an object of the present invention to provide novel herbicidally active compounds which allow better targeted control of undesirable plants than the known compounds.

We have found that this object is achieved by derivatives of 2,ω-diaminocarboxylic acids which are derivatized on both amino groups by aromatic or heteroaromatic carboxylic and/or sulfonic acids.

N-Acylated 2,ω-diaminocarboxylic acid compounds have been described by various documents of the prior art. The Japanese patent application J6 3060959 (Chem. Abstr. 109, (1998) 134966) describes N3-benzoyl-2,3-diaminopropionic acid and its antibacterial action. There have been various reports of oligopeptides comprising the amino acid building block 2,3-diaminopropionic acid, and of their antimicrobial and fungicidal action (see, for example, J. Shoji et al., J. Antibiot. 42, (1989), 869; R. Andruszkiewicz et al., Biochim. Biophys. Acta 828, (1985), 247; S. Fushiya et al., Tetrahedron Lett. 3071 (1980), R. Andruszkiewicz et al., J. Med. Chem. 30, (1987), 1715 and 33, (1990), 132, 2755).

P. Karrer et al., Helv. Chim. Acta 9, (1926) 314 describe the methyl and ethyl esters of doubly N-benzoylated derivatives of 2,3-diaminopropionic acid, of 2,4-diaminobutteric acid, or ornithine and of lysine in the context of the elucidation of the configuration of d-glutamic acid, of ornithine and of d-lysine.

Accordingly, the present invention relates to the use of 2,ω-diaminocarboxylic acid compounds of the formula I 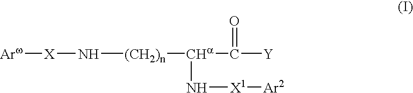
where
X, X¹ independently of one another are —CO— or —SO₂—,
Ar² and Ar^{ω} independently of one another are phenyl, naphthyl, mono- or bicyclic hetaryl having 5 to 10 ring atoms and 1, 2 or 3 hetero atoms, selected from nitrogen, oxygen and sulfur, where phenyl, naphthyl, mono- and bicyclic hetaryl may be unsubstituted or carry one, two, three, four or five substituents selected from the group consisting of halogen, hydroxyl, mercapto, nitro, cyano, CO₂H, HC(O), HC(O)O, C₁-C₁₂-alkyl, C₂-C₁₂-alkenyl, C₂-C₈-alkynyl, C₁-C₁₂-alkoxy, C₂-C₁₂-alkenyloxy, C₃-C₈-alkynyloxy, C₁-C₁₂-alkylthio, C₁-C₁₂-alkylsulfinyl, C₁-C₁₂-alkylsulfonyl, C₁-C₈-alkylcarbonyl, C₁-C₈-alkylcarbonyloxy, C₁-C₈-alkyloxycarbonyl, C₅-C₈-cycloalkyloxycarbonyl, C₁-C₈-haloalkyl, C₁-C₈-haloalkoxy, C₂-C₈-haloalkenyloxy, C₁-C₈-haloalkylthio, C₁-C₈-haloalkylsulfonyl, NH₂, NH-C₁-C₄-alkyl, N(C₁-C₄-alkyl)₂, C₁-C₆-alkoxy-C₁-C₄-alkyl, C₂-C₆-alkenyloxy-C₁-C₄-alkyl, C₃-C₄-alkynyloxy-C₁-C₄-alkyl, C₁-C₆-alkylthio-C₁-C₄-alkyl, C₂-C₆-alkenylthio-C₁-C₄-alkyl, C₁-C₈-alkylcarbonyl-C₁-C₄-alkyl, C₁-C₈-alkylcarbonyloxy-C₁-C₄-alkyl, C₁-C₈-alkyloxycarbonyl-C₁-C₄-alkyl, C₅-C₈-cycloalkyloxycarbonyl-C₁-C₄-alkyl, C₁-C₆-alkoxy-C₁-C₄-alkoxy, C₂-C₆-alkenyloxy-C₁-C₄-alkoxy, C₃-C₄-alkynyloxy-C₁-C₄-alkoxy, C₁-C₆-alkylthio-C₁-C₄-alkoxy, C₂-C₆-alkenylthio-C₁-C₄-alkoxy, C₁-C₈-alkylcarbonyl-C₁-C₄-alkoxy, C₁-C₈-alkylcarbonyloxy-C₁-C₄-alkoxy, C₁-C₈-alkyloxycarbonyl-C₁-C₄-alkoxy, C₅-C₈-cycloalkyloxycarbonyl-C₁-C₄-alkoxy, where substituents located at two adjacent carbon atoms of Ar² or Ar^{ω} may also form a C₃-C₅-alkylene chain which may be substituted and in which one or two nonadjacent methylene groups may be replaced by oxygen atoms;
n is 1, 2, 3, 4 or 5 and,
Y is —O—R¹ or,
R¹ is hydrogen, C₁-C₁₂-alkyl, C₂-C₁₂-alkenyl, C₃-C₈-alkynyl, C₁-C₁₂-haloalkyl, C₂-C₁₂-haloalkenyl, C₃-C₈-haloalkynyl, where in each case 1, 2 or 3 nonadjacent CH₂ groups may be replaced by oxygen, sulfur or an imino group,
   is C₃-C₈-cycloalkyl, C₃-C₈-cycloalkyl-C₁-C₄-alkyl, where the cycloalkyl moieties of the two last-mentioned groups may be partially or fully halogenated, may contain one or two double bonds and/or may carry one, two or three substituents selected from the group consisting of C₁-C₄-alkyl, hydroxyl, C₁-C₄-alkoxy, NH₂, NH—C₁-C₄-alkyl, N(C₁-C₄-alkyl)₂, where in the cycloalkyl moiety 1 or 2 nonadjacent CH₂ groups may be replaced by oxygen, sulfur or an imino group and the cycloalkyl moiety may have one or two carbonyl or thiocarbonyl groups as ring members,
   is phenyl-C₁-C₄-alkyl, phenoxy-C₁-C₄-alkyl or hetaryl-C₁-C₄-alkyl having 5 to 10 ring atoms and 1, 2 or 3 hetero atoms, selected from nitrogen, oxygen and sulfur, where the phenyl ring and the hetaryl ring of the three last-mentioned groups may be unsubstituted or carry one, two, three or four substituents selected from the group consisting of halogen, hydroxyl, amino, mercapto, C₁-C₄-alkyl, C₁-C₄-alkoxy, C₁-C₄-alkylcarbonyl, C₁-C₄-alkylcarbonyloxy, C₁-C₄-alkoxycarbonyl, C₁-C₄-haloalkyl, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, cyano and nitro,
R² is hydrogen, C₁-C₁₂-alkyl, C₂-C₁₂-alkenyl, C₃-C₈-alkynyl, C₁-C₁₂-haloalkyl, C₂-C₁₂-haloalkenyl, C₃-C₈-haloalkynyl, where in each case 1, 2 or 3 nonadjacent CH₂ groups may be replaced by oxygen, sulfur or an imino group,
   is C₃-C₈-cycloalkyl, C₃-C₈-cycloalkyl-C₁-C₄-alkyl, where the cycloalkyl moieties of the two last-mentioned groups may be partially or fully halogenated, may contain one or two double bonds and/or may carry, one, two or three substituents, selected from the group consisting of C₁-C₄-alkyl,hydroxyl, C₁-C₄-alkoxy, NH₂, NH—C₁-C₄-alkyl, N(C₁-C₄-alkyl)₂, where in the cycloalkyl moiety 1 or 2 nonadjacent CH₂ groups may be replaced by oxygen, sulfur or an imino group and the cycloalkyl moiety may contain one or two carbonyl or thiocarbonyl groups as ring members,
   is phenyl, phenyl-C₁-C₄-alkyl, phenoxy-C₁-C₄-alkyl, mono- or bicyclic hetaryl or hetaryl-C₁-C₄-alkyl having in each case 5 to 10 ring atoms and 1, 2 or 3 hetero atoms, selected from nitrogen, oxygen and sulfur, where the phenyl ring and the hetaryl ring of the five last-mentioned groups may be unsubstituted or carry one, two, three or four substituents selected from the group consisting of halogen, hydroxyl, amino, mercapto, C₁-C₄-alkyl, C₁-C₄-alkoxy, C₁-C₄-alkylcarbonyl, C₁-C₄-alkylcarbonyloxy, C₁-C₄-alkoxycarbonyl, C₁-C₄-haloalkyl, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, cyano and nitro,
   R³ has one of the meanings given for R² or together with R² and the nitrogen atom to which they are attached forms a saturated or unsaturated nitrogen heterocycle having 5, 6 or 7 ring atoms which may contain 1 or 2 additional hetero atoms, selected from nitrogen, oxygen and sulfur, and/or 1-carbonyl or thiocarbonyl groups as ring members and which may carry one, two, three or four substituents,
      or of agriculturally compatible salts of the compound I for controlling undesirable vegetation and as plant growth regulators (as herbicides).

The invention relates furthermore to the compounds of the formula I, except for compounds of the formula I in which Ar and Ar^{ω} are simultaneously unsubstituted phenyl, X and X¹ are C═O and Y is a group O—R¹ in which R¹ is H, CH₃ or C₂H₅, and to compositions comprising these compounds I. The invention furthermore relates to a process for preparing the compounds I.

Owing to the asymmetrically substituted α-carbon, the compounds of the formula I are present either as racemates, enantiomer mixtures or as pure enantiomers and, if they have chiral substituents on Ar^{ω}, Ar² or Y, they can also be present as diastereomer mixtures. Preference is given to those compounds of the formula I in which the α-carbon has the S configuration. Hereinbelow, these compounds are also referred to as S-enantiomers.

Suitable agriculturally useful salts are in particular the salts of those cations or the acid addition salts of those acids whose cations or anions, respectively, have no adverse effect on the herbicidal action of the compounds I. Thus, suitable cations are, in particular, the ions of the alkali metals, preferably sodium and potassium, of the alkaline earth metals, preferably calcium, magnesium and barium, and of the transition metals, preferably manganese, copper, zinc and iron, and the ammonium ion, which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reaction of I with an acid of the corresponding anion, preferably of hydrochloric acid, hydrobromic acid, sulfuric acid, phosphoric acid or nitric acid.

The organic moieties mentioned in the definition of the substituents on Ar² and Ar^{ω} or as radicals on cycloalkyl, phenyl or hetaryl rings are—like the term halogen-collective terms for individual enumerations of the individual group members. All carbon chains, i.e. all alkyl, haloalkyl, phenylalkyl, heterocyclylalkyl, alkoxy, haloalkoxy, alkylthio, haloalkylthio, alkylsulfinyl, haloalkylsulfinyl, alkylsulfonyl, haloalkylsulfonyl, alkenyl, haloalkenyl, alkenyloxy, alkenylthio, alkenylsulfinyl, alkenylsulfonyl, alkynyl and haloalkynyl moieties may be straight-chain or branched. Halogenated substituents preferably carry one to five identical or different halogen atoms. The term halogen denotes in each case fluorine, chlorine, bromine or iodine.

Examples of other meanings are:
C₁-C₄-alkyl: CH₃, C₂H₅, CH₂—C₂H₅, CH(CH₃)₂, n-butyl, CH(CH₃)—C₂H₅, CH₂—CH(CH₃)₂ or C(CH₃)₃;
C₁-C₄-haloalkyl: a C₁-C₄-alkyl radical as mentioned above which is partially or fully substituted by fluorine, chlorine, bromine and/or iodine, i.e., for example, CH₂F, CHF₂, CF₃, CH₂Cl, CH(Cl)₂, C(Cl)₃, chlorofluoromethyl, dichlorofluoromethyl, chlorodifluoromethyl, 2-fluoroethyl, 2-chloroethyl, 2-bromoethyl, 2-iodoethyl, 2,2-difluoroethyl, 2,2,2-trifluorooethyl, 2-chloro-2-fluoroethyl, 2-chloro-2,2-difluoroethyl, 2,2-dichloro-2-fluoroethyl, 2,2,2-trichloroethyl, C₂F₅, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, CH₂—C₂F₅, CF₂—C₂F₅, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl or nonafluorobutyl;
C₁-C₆-alkyl: a C₁-C₄-alkyl radical as mentioned above, or, for example, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl or 1-ethyl-2-methylpropyl, preferably CH₃, C₂H₅, CH₂—C₂H₅, CH(CH₃)₂, n-butyl, C(CH₃)₃, n-pentyl or n-hexyl;
C₁-C₁₂-alkyl: a C₁-C₆-alkyl radical as mentioned above and also saturated linear or branched hydrocarbon radicals having up to 12 carbons, such as n-heptyl, n-octyl, 2-ethylhexyl, n-decyl, n-undecyl and n-dodecyl.
C₁-C₆-haloalkyl: a C₁-C₆-alkyl radical as mentioned above which is partially or fully substituted by fluorine, chlorine, bromine and/or iodine, i.e., for example, one of the radicals mentioned under C₁-C₄-haloalkyl or 5-fluoro-1-pentyl, 5-chloro-1-pentyl, 5-bromo-1-pentyl, 5-iodine-1-pentyl, 5,5,5-trichloro-1-penyl, undecafluoropentyl, 6-fluoro-1-hexyl, 6-chloro-1-hexyl, 6-bromo-1-hexyl, 6-iodine-1-hexyl, 6,6,6-trichloro-1-hexyl or dodecafluorohexyl;
phenyl-C₁-C₄-alkyl: benzyl, 1-phenylethyl, 2-phenylethyl, 1-phenylprop-1-yl, 2-phenylprop-1-yl, 3-phenylprop-1-yl, 1-phenylbut-1-yl, 2-phenylbut-1-yl, 3-phenylbut-1-yl, 4-phenylbut-1-yl, 1-phenylbut-2-yl, 2-phenylbut-2-yl, 3-phenylbut-2-yl, 4-phenylbut-2-yl, 1-(benzyl)eth-1-yl, 1-(benzyl)-1-(methyl)eth-1-yl or 1-(benzyl)prop-1-yl, preferably benzyl or 2-phenylethyl;
phenoxy-C₁-C₄-alkyl: phenoxymethyl, 1-phenoxyethyl, 2-phenoxyethyl, 1-phenoxyprop-1-yl, 2-phenoxyprop-1-yl, 3-phenoxyprop-1-yl, 1-phenoxybut-1-yl, 2-phenoxybut-1-yl, 3-phenoxybut-1-yl, 4-phenoxybut-1-yl, 1-phenoxybut-2-yl, 2-phenoxybut-2-yl, 3-phenoxybut-2-yl, 4-phenoxybut-2-yl, 1-(phenoxymethyl)eth-1-yl, 1-(phenoxymethyl)-1-(methyl)-eth-1-yl or 1-(phenoxymethyl)prop-1-yl, preferably phenoxymethyl or 2-phenoxyethyl;
heterocyclyl-C₁-C₄-alkyl: heterocyclylmethyl, 1-heterocyclylethyl, 2-heterocyclylethyl, 1-heterocyclylprop-1-yl, 2-heterocyclylprop-1-yl, 3-heterocyclylprop-1-yl, 1-heterocyclylbut-1-yl, 2-heterocyclylbut-1-yl, 3-heterocyclylbut-1-yl, 4-heterocyclylbut-1-yl, 1-heterocyclylbut-2-yl, 2-heterocyclylbut-2-yl, 3-heterocyclylbut-2-yl, 3-heterocyclylbut-2-yl, 4-heterocyclylbut-2-yl, 1-(heterocyclyl-methyl)eth-1-yl, 1-(heterocyclylmethyl)-1-(methyl)eth-1-yl or 1-(heterocyclylmethyl)prop-1-yl, preferably heterocyclylmethyl or 2-heterocyclyl-ethyl;
C₁-C₄-alkoxy: OCH₃, OC₂H₅, OCH₂—C₂H₅, OCH(CH₃)₂, n-butoxy, OCH(CH₃)—C₂H₅, OCH₂—CH(CH₃)₂ or C(CH₃)₃, preferably OCH₃, OC₂H₅ or OCH(CH₃)₂;
C₁-C₄-haloalkoxy: a C₁-C₄-alkoxy radical as mentioned above which is partially or fully substituted by fluorine, chlorine, bromine and/or iodine, i.e., for example, OCH₂F, OCHF₂, OCF₃, OCH₂Cl, OCH(Cl)₂, OC(Cl)₃, chlorofluoromethoxy, dichlorofluormethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichlorethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichlorpropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy, preferably OCHF₂, OCF₃, dichlorofluoromethoxy, chlorodifluoromethoxy or 2,2,2-trifluoroethoxy;
C₁-C₈-alkoxy: a C₁-C₄-alkoxy radical as mentioned above or, for example, n-pentoxy, 1-methylbutoxy, 2-methylbutoxy, 3-methylbutoxy, 2,2-dimethylpropoxy, 1-ethylpropoxy, n-hexoxy, 1,1-dimethylpropoxy, 1,2-dimethylpropoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy, preferably OCH₃, OC₂H₅, OCH₂—C₂H₅, OCH(CH₃)₂, n-butoxy, OC(CH₃)₃, n-pentoxy or n-hexoxy;
C₁-C₈-haloalkoxy: a C₁-C₈-alkoxy radical as mentioned above which is partially or fully substituted by fluorine, chlorine, bromine and/or iodine, i.e., for example, one of the radicals mentioned under C₁-C₄-haloalkoxy or 5-fluoro-1-pentoxy, 5-chloro-1-pentoxy, 5-bromo-1-pentoxy, 5-iodo-1-pentoxy, 5,5,5-trichloro-1-pentoxy, undecafluoropentoxy, 6-fluoro-1-hexoxy, 6-chloro-1-hexoxy, 6-bromo-1-hexoxy, 6-iodo-1-hexoxy, 6,6,6-trichloro-1-hexoxy or dodecafluorohexoxy;
C₁-C₄-alkylthio: SCH₃, SC₂H₅, SCH₂—C₂H₅, SCH(CH₃)₂, n-butylthio, SCH(CH₃)—C₂H₅, SCH₂—CH(CH₃)₂ or SC(CH₃)₃, preferably SCH₃ or SC₂H₅;
C₁-C₄-haloalkylthio: a C₁-C₄-alkylthio radical as mentioned above which is partially or fully substituted by fluorine, chlorine, bromine and/or iodine, i.e., for example, SCH₂F, SCHF₂, SCF₃, SCH₂Cl, SCH(Cl)₂, SC(Cl)₃, chlorofluoromethylthio, dichlorofluoromethylthio, chlorodifluoromethylthio, 2-fluoroethylthio, 2-chloroethylthio, 2-bromoethylthio, 2-iodoethylthio, 2,2-difluoroethylthio, 2,2,2-trifluoroethylthio, 2-chloro-2-fluorethylthio, 2-chloro-2,2-difluoroethylthio, 2,2-dichloro-2-fluorethylthio, 2,2,2-trichlorethylthio, SC₂F₅, 2-fluoropropylthio, 3-fluoropropylthio, 2,2-difluoropropylthio, 2,3-difluoropropylthio, 2-chloropropylthio, 3-chloropropylthio, 2,3-dichloropropylthio, 2-bromopropylthio, 3-bromopropylthio, 3,3,3-trifluoropropylthio, 3,3,3-trichlorpropylthio, SCH₂—C₂F₅, SCF₂—C₂F₅, 1-(CH₂F)-2-fluoroethylthio, 1-(CH₂Cl)-2-chloroethylthio, 1-(CH₂Br)-2-bromoethylthio, 4-fluorobutylthio, 4-chlorobutylthio, 4-bromobutylthio or SCF₂—CF₂—C₂F₅, preferably SCHF₂, SCF₃, dichlorofluoromethylthio, chlorodifluoromethylthio or 2,2,2-trifluoroethylthio;
C₁-C₈-alkylthio: a C₁-C₄-alkylthio radical as mentioned above or, for example, n-pentylthio, 1-methylbutylthio, 2-methylbutylthio, 3-methylbutylthio, 2,2-dimethylpropylthio, 1-ethylpropylthio, n-hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio or 1-ethyl-2-methylpropylthio, preferably SCH₃, SC₂H₅, SCH₂—C₂H₅, SCH(CH₃)₂, n-butylthio, SC(CH₃)₃, n-pentylthio or n-hexylthio;
C₁-C₆-alkoxy-C₁-C₄-alkyl: C₁-C₄-alkyl which is substituted by C₁-C₆-alkoxy—as mentioned above—, i.e., for example, CH₂—OCH₃, CH₂—OC₂H₅, n-propoxymethyl, CH₂—OCH(CH₃)₂, n-butoxymethyl, (1-methylpropoxy)methyl, (2-methylpropoxy)methyl, CH₂—OC(CH₃)₃, 2-(methoxy)ethyl, 2-(ethoxy)ethyl, 2-(n-propoxy)ethyl, 2-(1-methylethoxy)ethyl, 2-(n-butoxy)ethyl, 2-(1-methylpropoxy)ethyl, 2-(2-methylpropoxy)ethyl, 2-(1,1-dimethylethoxy)ethyl, 2-(methoxy)propyl, 2-(ethoxy)propyl, 2-(n-propoxy)propyl, 2-(1-methylethoxy)propyl, 2-(n-butoxy)-propyl, 2-(1-methylpropoxy)propyl, 2-(2-methylpropoxy)propyl, 2-(1,1-dimethylethoxy)propyl, 3-(methoxy)propyl, 3-(ethoxy)-propyl, 3-(n-propoxy)propyl, 3-(1-methylethoxy)propyl, 3-(n-butoxy)propyl, 3-(1-methylpropoxy)propyl, 3-(2-methylpropoxy)propyl, 3-(1,1-dimethylethoxy)propyl, 2-(methoxy)butyl, 2-(ethoxy)butyl, 2-(n-propoxy)butyl, 2-(1-methylethoxy)butyl, 2-(n-butoxy)butyl, 2-(1-methylpropoxy)butyl, 2-(2-methylpropoxy)butyl, 2-(1,1-dimethylethoxy)butyl, 3-(methoxy)butyl, 3-(ethoxy)butyl, 3-(n-propoxy)butyl, 3-(1-methylethoxy)butyl, 3-(n-butoxy)butyl, 3-(1-methylpropoxy)butyl, 3-(2-methylpropoxy)butyl, 3-(1,1-dimethylethoxy)butyl, 4-(methoxy)butyl, 4-(ethoxy)butyl, 4-(n-propoxy)butyl, 4-(1-methylethoxy)butyl, 4-(n-butoxy)butyl, 4-(1-methylpropoxy)butyl, 4-(2-methylpropoxy)butyl or 4-(1,1-dimethylethoxy)butyl, preferably CH₂—OCH₃, CH₂—OC₂H₅, 2-(OCH₃)ethyl or 2-(OC₂H₅)ethyl;
C₁-C₆-alkoxy-C₁-C₄-alkoxy-C₁-C₄-alkyl: C₁-C₄-alkoxy-C₁-C₄-alkyl which is substituted by C₁-C₆-alkoxy—as mentioned above—, i.e., for example, CH₂CH₂—OCH₂CH₂—OCH₃, CH₂CH₂—OCH₂CH₂—OC₂H₅, n-propoxyethoxyethyl, CH₂CH₂—OCH₂CH₂—OCH(CH₃)₂, n-butoxyethoxyethyl, (1-methylpropoxy)ethoxyethyl, (2-methylpropoxy)ethoxyethyl, CH₂CH₂—OCH₂CH₂—OC(CH₃)₃, 2-(methoxy)ethoxypropyl, 2-(ethoxy)ethoxypropyl, 2-(n-propoxy)ethoxypropyl, 2-(1-methylethoxy)ethoxypropyl, 2-(n-butoxy)ethoxypropyl, 2-(1-methylpropoxy)ethoxypropyl, 2-(2-methylpropoxy)ethoxypropyl, 2-(1,1-dimethylethoxy)ethoxypropyl, 3-(methoxy)propoxypropyl, 3-(ethoxy)propoxypropyl, 3-(n-propoxy)propoxypropyl, 3-(1-methylethoxy)propoxypropyl, 3-(n-butoxy)propoxypropyl, 3-(1-methylpropoxy)propoxypropyl, 3-(2-methylpropoxy)propoxypropyl, 3-(1,1-dimethylethoxy)propoxypropyl;
C₁-C₄-haloalkoxy-C₁-C₄-alkyl: C₁-C₄-alkyl which is substituted by C₁-C₄-haloalkoxy as mentioned above, i.e., for example, 2-(OCHF₂)ethyl, 2-(OCF₃)ethyl or 2-(OC₂F₅)ethyl;
C₁-C₆-alkylthio-C₁-C₄-alkyl: C₁-C₄-alkyl which is substituted by C₁-C₄-alkylthio—as mentioned above—, i.e., for example, CH₂—SCH₃, CH₂—SC₂H₅, n-propylthiomethyl, CH₂—SCH(CH₃)₂, n-butylthiomethyl, (1-methylpropylthio)methyl, (2-methylpropylthio)methyl, CH₂—SC(CH₃)₃, 2-(methylthio)ethyl, 2-(ethylthio)ethyl, 2-(n-propylthio)ethyl, 2-(1-methylethylthio)ethyl, 2-(n-butylthio)ethyl, 2-(1-methylpropylthio)ethyl, 2-(2-methylpropylthio)ethyl, 2-(1,1-dimethylethylthio)ethyl, 2-(methylthio)propyl, 2-(ethylthio)propyl, 2-(n-propylthio)propyl, 2-(1-methylethylthio)propyl, 2-(n-butylthio)propyl, 2-(1-methylpropylthio)propyl, 2-(2-methylpropylthio)propyl, 2-(1,1-dimethylethylthio)propyl, 3-(methylthio)propyl, 3-(ethylthio)propyl, 3-(n-propylthio)propyl, 3-(1-methylethylthio)propyl, 3-(n-butylthio)propyl, 3-(1-methylpropylthio)-propyl, 3-(2-methylpropylthio)propyl, 3-(1,1-dimethylethylthio)propyl, 2-(methylthio)butyl, 2-(ethylthio)butyl, 2-(n-propylthio)butyl, 2-(1-methylethylthio)butyl, 2-(n-butylthio)butyl, 2-(1-methylpropylthio)butyl, 2-(2-methylpropylthio)butyl, 2-(1,1-dimethylethylthio)butyl, 3-(methylthio)butyl, 3-(ethylthio)butyl, 3-(n-propylthio)butyl, 3-(1-methylethylthio)butyl, 3-(n-butylthio)butyl, 3-(1-methylpropylthio)butyl, 3-(2-methylpropylthio)butyl, 3-(1,1-dimethylethylthio)butyl, 4-(methylthio)butyl, 4-(ethylthio)butyl, 4-(n-propylthio)butyl, 4-(1-methylethylthio)butyl, 4-(n-butylthio)-butyl, 4-(1-methylpropylthio)butyl, 4-(2-methylpropylthio)butyl or 4-(1,1-dimethylethylthio)butyl, vorzugsweise CH₂—SCH₃, CH₂—SC₂H₅, 2-(SCH₃)ethyl or 2-(SC₂H₅)ethyl;
C₁-C₄-haloalkylthio-C₁-C₄-alkyl: C₁-C₄-alkyl which is substituted by C₁-C₄-haloalkylthio as mentioned above, i.e., for example, 2-(SCHF₂)ethyl, 2-(SCF₃)ethyl or 2-(SC₂F₅)ethyl;
amino-C₁-C₄-alkyl: CH₂NH₂, 1-aminoethyl, 2-aminoethyl, 1-aminoprop-1-yl, 2-aminoprop-1-yl, 3-aminoprop-1-yl, 1-amino-but-1-yl, 2-aminobut-1-yl, 3-aminobut-1-yl, 4-aminobut-1-yl, 1-aminobut-2-yl, 2-aminobut-2-yl, 3-aminobut-2-yl, 4-amino-but-2-yl, 1-(CH₂NH₂)eth-1-yl, 1-(CH₂NH₂)-1-(CH₃)eth-1-yl or 1-(CH₂NH₂)prop-1-yl;
C₁-C₄-alkylamino-C₁-C₄-alkyl: C₁-C₄-alkyl which is substituted by C₁-C₄-alkylamino such as methylamino, ethylamino, n- or isopropylamino, i.e., for example, CH₂CH₂—NH—CH₃, CH₂CH₂—NH—C₂H₅, CH₂CH₂—NH-(n-C₃H₇), CH₂CH₂—NH-(i-C₃H₇), CH₂CH₂CH₂—NH—CH₃, CH₂CH₂CH₂—NH—C₂H₅, CH₂CH₂CH₂—NH-(n-C₃H₇) or CH₂CH₂CH₂—NH— (i-C₃H₇);
di(C₁-C₄-alkyl)amino: N(CH₃)₂, N(C₂H₅)₂, N,N-dipropylamino, N,N-di-(1-methylethyl)amino, N,N-dibutylamino, N,N-di-(1-methylpropyl)amino, N,N-di-(2-methylpropyl)amino, N,N-di-(1,1-dimethylethyl)amino, N-ethyl-N-methylamino, N-methyl-N-propylamino, N-methyl-N-(1-methylethyl)amino, N-butyl-N-methylamino, N-methyl-N-(1-methylpropyl)amino, N-methyl-N-(2-methylpropyl)amino, N-(1,1-dimethylethyl)-N-methylamino, N-ethyl-N-propylamino, N-ethyl-N-(1-methylethyl)amino, N-butyl-N-ethylamino, N-ethyl-N-(1-methylpropyl)amino, N-ethyl-N-(2-methylpropyl)amino, N-ethyl-N-(1,1-dimethylethyl)amino, N-(1-methylethyl)-N-propylamino, N-butyl-N-propylamino, N-(1-methylpropyl)-N-propylamino, N-(2-methylpropyl)-N-propylamino, N-(1,1-dimethylethyl)-N-propylamino, N-butyl-N-(1-methylethyl)amino, N-(1-methylethyl)-N-(1-methylpropyl)amino, N-(1-methylethyl)-N-(2-methylpropyl)amino, N-(1,1-dimethylethyl)-N-(1-methylethyl)amino, N-butyl-N-(1-methylpropyl)amino, N-butyl-N-(2-methylpropyl)amino, N-butyl-N-(1,1-dimethylethyl)-amino, N-(1-methylpropyl)-N-(2-methylpropyl)amino, N-(1,1-dimethylethyl)-N-(1-methylpropyl)amino or N-(1,1-dimethylethyl)-N-(2-methylpropyl)amino, preferably N(CH₃)₂ or N(C₂H₅)₂;
di(C₁-C₄-alkyl)amino-C₁-C₄-alkyl: C₁-C₄-alkyl which is substituted by di(C₁-C₄-alkyl)amino as mentioned above, i.e., for example, CH₂N(CH₃)₂, CH₂N(C₂H₅)₂, N,N-dipropylaminomethyl, N,N-di[CH(CH₃)₂]aminomethyl, N,N-dibutylaminomethyl, N,N-di-(1-methylpropyl)aminomethyl, N,N-di(2-methylpropyl)aminomethyl, N,N-di[C(CH₃)₃]aminomethyl, N-ethyl-N-methylaminomethyl, N-methyl-N-propylaminomethyl, N-methyl-N-[CH(CH₃)₂]aminomethyl, N-butyl-N-methylaminomethyl, N-methyl-N-(1-methylpropyl)aminomethyl, N-methyl-N-(2-methylpropyl)aminomethyl, N-[C(CH₃)₃]-N-methylaminomethyl, N-ethyl-N-propylaminomethyl, N-ethyl-N-[CH(CH₃)₂]aminomethyl, N-butyl-N-ethylaminomethyl, N-ethyl-N-(1-methylpropyl)aminomethyl, N-ethyl-N-(2-methylpropyl)aminomethyl, N-ethyl-N-[C(CH₃)₃]aminomethyl, N-[CH(CH₃)₂]-N-propylaminomethyl, N-butyl-N-propylaminomethyl, N-(1-methylpropyl)-N-propylaminomethyl, N-(2-methylpropyl)-N-propylaminomethyl, N-[C(CH₃)₃]-N-propylaminomethyl, N-butyl-N-(1-methylethyl)-aminomethyl, N-[CH(CH₃)₂]-N-(1-methylpropyl)aminomethyl, N-[CH(CH₃)₂]-N-(2-methylpropyl)aminomethyl, N-[C(CH₃)₃]-N-[CH(CH₃)₂]aminomethyl, N-butyl-N-(1-methylpropyl)aminomethyl, N-butyl-N-(2-methylpropyl)aminomethyl, N-butyl-N-[C(CH₃)₃]-aminomethyl, N-(1-methylpropyl)-N-(2-methylpropyl)aminomethyl, N-[C(CH₃)₃]-N-(1-methylpropyl)aminomethyl, N-[C(CH₃)₃]-N-(2-methylpropyl)aminomethyl, N,N-dimethylaminoethyl, N,N-diethylaminoethyl, N,N-di(n-propyl)aminoethyl, N,N-di-[CH(CH₃)₂]aminoethyl, N,N-dibutylaminoethyl, N,N-di(1-methylpropyl)aminoethyl, N,N-di(2-methylpropyl)aminoethyl, N,N-di-[C(CH₃)₃]aminoethyl, N-ethyl-N-methylaminoethyl, N-methyl-N-propylaminoethyl, N-methyl-N-[CH(CH₃)₂]aminoethyl, N-butyl-N-methylaminoethyl, N-methyl-N-(1-methylpropyl)aminoethyl, N-methyl-N-(2-methylpropyl)aminoethyl, N-[C(CH₃)₃]-N-methylaminoethyl, N-ethyl-N-propylaminoethyl, N-ethyl-N-[CH(CH₃)₂]aminoethyl, N-butyl-N-ethylaminoethyl, N-ethyl-N-(1-methylpropyl)aminoethyl, N-ethyl-N-(2-methylpropyl)aminoethyl, N-ethyl-N-[C(CH₃)₃]aminoethyl, N-[CH(CH₃)₂]-N-propylaminoethyl, N-butyl-N-propylaminoethyl, N-(1-methylpropyl)-N-propylaminoethyl, N-(2-methylpropyl)-N-propylaminoethyl, N-[C(CH₃)₃]-N-propylaminoethyl, N-butyl-N-[CH(CH₃)₂]aminoethyl, N-[CH(CH₃)₂]-N-(1-methylpropyl)aminoethyl, N-[CH(CH₃)₂]-N-(2-methylpropyl)aminoethyl, N-[C(CH₃)₃]-N-[CH(CH₃)₂]aminoethyl, N-butyl-N-(1-methylpropyl)aminoethyl, N-butyl-N-(2-methylpropyl)aminoethyl, N-butyl-N-[C(CH₃)₃]aminoethyl, N-(1-methylpropyl)-N-(2-methylpropyl)aminoethyl, N-[C(CH₃)₃]-N-(1-methylpropyl)aminoethyl or N-[C(CH₃)₃]-N-(2-methylpropyl)aminoethyl, in particular N,N-dimethylaminoethyl or N,N-diethylaminoethyl;
C₁-C₈-alkylsulfinyl: a C₁-C₄-alkylsulfinyl radical such as SO—CH₃, SO—C₂H₅, SO—CH₂—C₂H₅, SO—CH(CH₃)₂, SO-(n-C₄H₉), SO—CH(CH₃)—C₂H₅, SO—CH₂—CH(CH₃)₂ or SO—C(CH₃)₃, or, for example, SO-(n-C₅H₁₁), 1-methylbutyl-SO, 2-methylbutyl-SO, 3-methylbutyl-SO, 2,2-dimethylpropyl-SO, 1-ethylpropyl-SO, n-hexyl-SO, 1,1-dimethylpropyl-SO, 1,2-dimethylpropyl-SO, 1-methylpentyl-SO, 2-methylpentyl-SO, 3-methylpentyl-SO, 4-methylpentyl-SO, 1,1-dimethylbutyl-SO, 1,2-dimethylbutyl-SO, 1,3-dimethylbutyl-SO, 2,2-dimethylbutyl-SO, 2,3-dimethylbutyl-SO, 3,3-dimethylbutyl-SO, 1-ethylbutyl-SO, 2-ethylbutyl-SO, 1,1,2-trimethylpropyl-SO, 1,2,2-trimethylpropyl-SO, 1-ethyl-1-methylpropyl-SO or 1-ethyl-2-methylpropyl-SO, preferably SO—CH₃, SO—C₂H₅, SO—CH₂—C₂H₅, SO—CH(CH₃)₂, SO-(n-C₄H₉), SO—C(CH₃)₃, SO-(n-C₅H₁₁) or SO-(n-C₆H₁₃);
C₁-C₄-alkylsulfonyl: SO₂—CH₃, SO₂—C₂H₅, SO₂—CH₂—C₂H₅, SO₂—CH(CH₃)₂, n-butylsulfonyl, SO₂—CH(CH₃)—C₂H₅, SO₂—CH₂—CH(CH₃)₂ or SO₂—C(CH₃)₃, preferably SO₂—CH₃ or SO₂—C₂H₅;
C₁-C₄-haloalkylsulfonyl: a C₁-C₄-alkylsulfonyl radical—as mentioned above—which is partially or fully substituted by fluorine, chlorine, bromine and/or iodine, i.e., for example, SO₂—CH₂F, SO₂—CHF₂, SO₂—CF₃, SO₂—CH₂Cl, SO₂—CH(Cl)₂, SO₂—C(Cl)₃, chlorofluoromethylsulfonyl, dichlorofluoromethylsulfonyl, chlorodifluoromethylsulfonyl, 2-fluoroethylsulfonyl, 2-chloroethylsulfonyl, 2-bromoethylsulfonyl, 2-iodoethylsulfonyl, 2,2-difluoroethylsulfonyl, 2,2,2-trifluoroethylsulfonyl, 2-chloro-2-fluoroethylsulfonyl, 2-chloro-2,2-difluoroethylsulfonyl, 2,2-dichloro-2-fluoroethylsulfonyl, 2,2,2-trichloroethylsulfonyl, SO₂—C₂F₅, 2-fluoropropylsulfonyl, 3-fluoropropylsulfonyl, 2,2-difluoropropylsulfonyl, 2,3-difluoropropylsulfonyl, 2-chloropropylsulfonyl, 3-chloropropylsulfonyl, 2,3-dichloropropylsulfonyl, 2-bromopropylsulfonyl, 3-bromopropylsulfonyl, 3,3,3-trifluoropropylsulfonyl, 3,3,3-trichloropropylsulfonyl, SO₂—CH₂—C₂F₅, SO₂—CF₂—C₂F₅, 1-(fluoromethyl)-2-fluoroethylsulfonyl, 1-(chloromethyl)-2-chloroethylsulfonyl, 1-(bromomethyl)-2-bromoethylsulfonyl, 4-fluorobutylsulfonyl, 4-chlorobutylsulfonyl, 4-bromobutylsulfonyl or nonafluorobutylsulfonyl, preferably SO₂—CH₂Cl, SO₂—CF₃ or 2,2,2-trifluoroethylsulfonyl;
C₁-C₈-alkylsulfonyl: a C₁-C₄-alkylsulfonyl radical as mentioned above, or, for example, SO₂-(n-C₅H₁₁), 1-methylbutyl-SO₂, 2-methylbutyl-SO₂, 3-methylbutyl-SO₂, 2,2-dimethylpropyl-SO₂, 1-ethylpropyl-SO₂, n-hexyl-SO₂, 1,1-dimethylpropyl-SO₂, 1,2-dimethylpropyl-SO₂, 1-methylpentyl-SO₂, 2-methylpentyl-SO₂, 3-methylpentyl-SO₂, 4-methylpentyl-SO₂, 1,1-dimethylbutyl-SO₂, 1,2-dimethylbutyl-SO₂, 1,3-dimethylbutyl-SO₂, 2,2-dimethylbutyl-SO₂, 2,3-dimethylbutyl-SO₂, 3,3-dimethylbutyl-SO₂, 1-ethylbutyl-SO₂, 2-ethylbutyl-SO₂, 1,1,2-trimethylpropyl-SO₂, 1,2,2-trimethylpropyl-SO₂, 1-ethyl-1-methylpropyl-SO₂ or 1-ethyl-2-methylpropyl-SO₂, preferably SO₂—CH₃, SO₂—C₂H₅, SO₂—CH₂—C₂H₅, SO₂—CH(CH₃)₂, SO₂-(n-C₄H₉), SO₂—C(CH₃)₃, SO₂-(n-C₅H₁₁) or SO₂-(n-C₆H₁₃);
C₁-C₆-alkylsulfinyl-C₁-C₄-alkyl, C₁-C₆-alkylsulfonyl-C₁-C₄-alkyl: C₁-C₄-alkyl which is monosubstituted by C₁-C₆-alkylsulfinyl or C₁-C₆-alkylsulfonyl;
(C₁-C₈-alkyl)carbonyl: a C₁-C₈-alkyl radical which is attached via a carbonyl group, such as CO—CH₃, CO—C₂H₅, CO—CH₂—C₂H₅, CO—CH(CH₃) 2, n-butylcarbonyl, CO—CH(CH₃)—C₂H₅, CO—CH₂—CH(CH₃)₂ or CO—C(CH₃)₃, preferably CO—CH₃ or CO—C₂H₅;
(C₁-C₈-haloalkyl)carbonyl: a (C₁-C₈-alkyl)carbonyl radical—as mentioned above—which is partially or fully substituted by fluorine, chlorine, bromine and/or iodine, i.e., for example, CO—CH₂F, CO—CHF₂, CO—CF₃, CO—CH₂Cl, CO—CH(Cl)₂, CO—C(Cl)₃, chlorofluoromethylcarbonyl, dichlorofluoromethylcarbonyl, chlorodifluoromethylcarbonyl, 2-fluoroethylcarbonyl, 2-chloroethylcarbonyl, 2-bromoethylcarbonyl, 2-iodoethylcarbonyl, 2,2-difluoroethylcarbonyl, 2,2,2-trifluoroethylcarbonyl, 2-chloro-2-fluoroethylcarbonyl, 2-chloro-2,2-difluoroethylcarbonyl, 2,2-dichloro-2-fluoroethylcarbonyl, 2,2,2-trichloroethylcarbonyl, CO—C₂F₅, 2-fluoropropylcarbonyl, 3-fluoropropylcarbonyl, 2,2-difluoropropylcarbonyl, 2,3-difluoropropylcarbonyl, 2-chloropropylcarbonyl, 3-chloropropylcarbonyl, 2,3-dichloropropylcarbonyl, 2-bromopropylcarbonyl, 3-bromopropylcarbonyl, 3,3,3-trifluoropropylcarbonyl, 3,3,3-trichloropropylcarbonyl, CO—CH₂—C₂F₅, CO—CF₂—C₂F₅, 1-(CH₂F)-2-fluoroethylcarbonyl, 1-(CH₂Cl)-2-chloroethylcarbonyl, 1-(CH₂Br)-2-bromoethylcarbonyl, 4-fluorobutylcarbonyl, 4-chlorobutylcarbonyl, 4-bromobutylcarbonyl or nonafluorobutylcarbonyl, preferably CO—CF₃, CO—CH₂Cl or 2,2,2-trifluoroethylcarbonyl;
(C₁-C₈-alkyl)carbonyloxy: a (C₁-C₈-alkyl)carbonyl radical, which is attached via an oxygen atom, such as O—CO—CH₃, O—CO—C₂H₅, O—CO—CH₂—C₂H₅, O—CO—CH(CH₃) 2, O—CO—CH₂—CH₂—C₂H₅, O—CO—CH(CH₃)—C₂H₅, O—CO—CH₂—CH(CH₃)₂ or O—CO—C(CH₃)₃, preferably O—CO—CH₃ or O—CO—C₂H₅;
(C₁-C₈-haloalkyl)carbonyloxy: a (C₁-C₈-alkyl)carbonyloxy radical—as mentioned above—which is partially or fully substituted by fluorine, chlorine, bromine and/or iodine, i.e., for example, O—CO—CH₂F, O—CO—CHF₂, O—CO—CF₃, O—CO—CH₂Cl, O—CO—CH(Cl)₂, O—CO—C(Cl)₃, chlorofluoromethylcarbonyloxy, dichlorofluoromethylcarbonyloxy, chlorodifluoromethylcarbonyloxy, 2-fluoroethylcarbonyloxy, 2-chloroethylcarbonyloxy, 2-bromoethylcarbonyloxy, 2-iodoethylcarbonyloxy, 2,2-difluoroethylcarbonyloxy, 2,2,2-trifluoroethylcarbonyloxy, 2-chloro-2-fluoroethylcarbonyloxy, 2-chloro-2,2-difluoroethylcarbonyloxy, 2,2-dichloro-2-fluoroethylcarbonyloxy, 2,2,2-trichloroethylcarbonyloxy, O—CO—C₂F₅, 2-fluoropropylcarbonyloxy, 3-fluoropropylcarbonyloxy, 2,2-difluoropropylcarbonyloxy, 2,3-difluoropropylcarbonyloxy, 2-chloropropylcarbonyloxy, 3-chloropropylcarbonyloxy, 2,3-dichloropropylcarbonyloxy, 2-bromopropylcarbonyloxy, 3-bromopropylcarbonyloxy, 3,3,3-trifluoropropylcarbonyloxy, 3,3,3-trichloropropylcarbonyloxy, O—CO—CH₂—C₂F₅, O—CO—CF₂—C₂F₅, 1-(CH₂F)-2-fluoroethylcarbonyloxy, 1-(CH₂Cl)-2-chloroethylcarbonyloxy, 1-(CH₂Br)-2-bromoethylcarbonyloxy, 4-fluorobutylcarbonyloxy, 4-chlorobutylcarbonyloxy, 4-bromobutylcarbonyloxy or nonafluorobutylcarbonyloxy, preferably O—CO—CF₃, O—CO—CH₂Cl or 2,2,2-trifluoroethylcarbonyloxy;
(C₁-C₈-alkoxy)carbonyl: a C₁-C₈-alkoxy radical which is attached via a carbonyl group, for example CO—OCH₃, CO—OC₂H₅, CO—OCH₂—C₂H₅, CO—OCH(CH₃)₂, n-butoxycarbonyl, CO—OCH(CH₃)—C₂H₅, CO—OCH₂—CH(CH₃)₂ or CO—OC(CH₃)₃, preferably CO—OCH₃ or CO—OC₂H₅;
(C₁-C₆-alkoxy)carbonyl: one of the (C₁-C₄-alkoxy)carbonyl radicals mentioned above, or, for example, n-pentoxy-CO, 1-methylbutoxy-CO, 2-methylbutoxy-CO, 3-methylbutoxy-CO, 2,2-dimethylpropoxy-CO, 1-ethylpropoxy-CO, n-hexoxy-CO, 1,1-dimethylpropoxy-CO, 1,2-dimethylpropoxy-CO, 1-methylpentoxy-CO, 2-methylpentoxy-CO, 3-methylpentoxy-CO, 4-methylpentoxy-CO, 1,1-dimethylbutoxy-CO, 1,2-dimethylbutoxy-CO, 1,3-dimethylbutoxy-CO, 2,2-dimethylbutoxy-CO, 2,3-dimethylbutoxy-CO, 3,3-dimethylbutoxy-CO, 1-ethylbutoxy-CO, 2-ethylbutoxy-CO, 1,1,2-trimethylpropoxy-CO, 1,2,2-trimethylpropoxy-CO, 1-ethyl-1-methylpropoxy-CO or 1-ethyl-2-methylpropoxy-CO, preferably CO—OCH₃, CO—OC₂H₅, CO—OCH₂—C₂H₅, CO—OCH(CH₃)₂, n-butoxy-CO, CO—OC(CH₃)₃, n-pentoxy-CO or n-hexoxy-CO;
C₂-C₁₂-alkenyl: a mono- or polyethylenically unsaturated radical having 2 to 12, preferably 2 to 8 and in particular 2 to 6 carbon atoms, such as ethenyl, prop-1-en-1-yl, allyl, 1-methylethenyl, 1-buten-1-yl, 1-buten-2-yl, 1-buten-3-yl, 2-buten-1-yl, 1-methylprop-1-en-1-yl, 2-methylprop-1-en-1-yl, 1-methylprop-2-en-1-yl, 2-methylprop-2-en-1-yl, n-penten-1-yl, n-penten-2-yl, n-penten-3-yl, n-penten-4-yl, 1-methylbut-1-en-1-yl, 2-methylbut-1-en-1-yl, 3-methylbut-1-en-1-yl, 1-methylbut-2-en-1-yl, 2-methylbut-2-en-1-yl, 3-methylbut-2-en-1-yl, 1-methylbut-3-en-1-yl, 2-methylbut-3-en-1-yl, 3-methylbut-3-en-1-yl, 1,1-dimethylprop-2-en-1-yl, 1,2-dimethylprop-1-en-1-yl, 1,2-dimethylprop-2-en-1-yl, 1-ethylprop-1-en-2-yl, 1-ethylprop-2-en-1-yl, n-hex-1-en-1-yl, n-hex-2-en-1-yl, n-hex-3-en-1-yl, n-hex-4-en-1-yl, n-hex-5-en-1-yl, 1-methylpent-1-en-1-yl, 2-methylpent-1-en-1-yl, 3-methylpent-1-en-1-yl, 4-methylpent-1-en-1-yl, 1-methylpent-2-en-1-yl, 2-methylpent-2-en-1-yl, 3-methylpent-2-en-1-yl, 4-methylpent-2-en-1-yl, 1-methylpent-3-en-1-yl, 2-methylpent-3-en-1-yl, 3-methylpent-3-en-1-yl, 4-methylpent-3-en-1-yl, 1-methylpent-4-en-1-yl, 2-methylpent-4-en-1-yl, 3-methylpent-4-en-1-yl, 4-methylpent-4-en-1-yl, 1,1-dimethylbut-2-en-1-yl, 1,1-dimethylbut-3-en-1-yl, 1,2-dimethylbut-1-en-1-yl, 1,2-dimethylbut-2-en-1-yl, 1,2-dimethylbut-3-en-1-yl, 1,3-dimethylbut-1-en-1-yl, 1,3-dimethylbut-2-en-1-yl, 1,3-dimethylbut-3-en-1-yl, 2,2-dimethylbut-3-en-1-yl, 2,3-dimethylbut-1-en-1-yl, 2,3-dimethylbut-2-en-1-yl, 2,3-dimethylbut-3-en-1-yl, 3,3-dimethylbut-1-en-1-yl, 3,3-dimethylbut-2-en-1-yl, 1-ethylbut-1-en-1-yl, 1-ethylbut-2-en-1-yl, 1-ethylbut-3-en-1-yl, 2-ethylbut-1-en-1-yl, 2-ethylbut-2-en-1-yl, 2-ethylbut-3-en-1-yl, 1,1,2-trimethylprop-2-en-1-yl, 1-ethyl-1-methylprop-2-en-1-yl, 1-ethyl-2-methylprop-1-en-1-yl or 1-ethyl-2-methylprop-2-en-1-yl;
C₂-C₁₂-alkenyloxy: a C₂-C₁₂-alkenyl radical as mentioned above which is attached via an oxygen atom;
C₂-C₁₂-alkenyloxy-C₁-C₄-alkyl: C₁-C₄-alkyl which carries a C₂-C₁₂-alkenyloxy radical;
C₂-C₁₂-alkenylthio: a C₂-C₁₂-alkenyl radical as mentioned above which is attached via a sulfur atom;
C₂-C₁₂-alkenylthio-C₁-C₄-alkyl: a C₁-C₄-alkyl which carries a C₂-C₁₂-alkenylthio radical;
C₃-C₈-haloalkenyl: alkenyl having 2 to 8 carbons as mentioned above, which is partially or fully substituted by fluorine, chlorine, bromine and/or iodine, i.e., for example, 2-chloroallyl, 3-chloroallyl, 2,3-dichloroallyl, 3,3-dichloroallyl, 2,3,3-trichloroallyl, 2,3-dichlorobut-2-enyl, 2-bromoallyl, 3-bromoallyl, 2,3-dibromoallyl, 3,3-dibromoallyl, 2,3,3-tribromoallyl or 2,3-dibromobut-2-enyl;
C₃-C₈-alkynyl: prop-1-yn-1-yl, prop-2-yn-1-yl, n-but-1-yn-1-yl, n-but-1-yn-3-yl, n-but-1-yn-4-yl, n-but-2-yn-1-yl, n-pent-1-yn-1-yl, n-pent-1-yn-3-yl, n-pent-1-yn-4-yl, n-pent-1-yn-5-yl, n-pent-2-yn-1-yl, n-pent-2-yn-4-yl, n-pent-2-yn-5-yl, 3-methylbut-1-yn-3-yl, 3-methylbut-1-yn-4-yl, n-hex-1-yn-1-yl, n-hex-1-yn-3-yl, n-hex-1-yn-4-yl, n-hex-1-yn-5-yl, n-hex-1-yn-6-yl, n-hex-2-yn-1-yl, n-hex-2-yn-4-yl, n-hex-2-yn-5-yl, n-hex-2-yn-6-yl, n-hex-3-yn-1-yl, n-hex-3-yn-2-yl, 3-methylpent-1-yn-1-yl, 3-methylpent-1-yn-3-yl, 3-methylpent-1-yn-4-yl, 3-methylpent-1-yn-5-yl, 4-methylpent-1-yn-1-yl, 4-methylpent-2-yn-4-yl or 4-methylpent-2-yn-5-yl, preferably prop-2-yn-1-yl;
C₃-C₈-haloalkynyl: C₃-C₈-alkynyl as mentioned above which is partially or fully substituted by fluorine, chlorine, bromine and/or iodine, i.e., for example, 1,1-difluoroprop-2-yn-1-yl, 4-fluorobut-2-yn-1-yl, 4-chlorobut-2-yn-1-yl, 1,1-difluorobut-2-yn-1-yl, 5-fluoropent-3-yn-1-yl or 6-fluorohex-4-yn-1-yl;
C₂-C₆-alkenyloxy-C₁-C₄-alkyl: C₁-C₄-alkyl which is substituted by C₂-C₆-alkenyloxy such as allyloxy, but-1-en-3-yloxy, but-1-en-4-yloxy, but-2-en-1-yloxy, 1-methylprop-2-enyloxy or 2-methylprop-2-enyloxy, i.e., for example, allyloxymethyl, 2-allyloxyethyl or but-1-en-4-yloxymethyl, in particular 2-allyloxyethyl;
C₃-C₄-alkynyloxy-C₁-C₄-alkyl: C₁-C₄-alkyl which is substituted by C₃-C₄-alkynyloxy such as propargyloxy, but-1-yn-3-yloxy, but-1-yn-4-yloxy, but-2-yn-1-yloxy, 1-methylprop-2-ynyloxy or 2-methylprop-2-ynyloxy, preferably by propargyloxy, i.e., for example, propargyloxymethyl or 2-propargyloxyethyl, in particular 2-propargyloxyethyl;
C₃-C₈-cycloalkyl: cyclopropyl, cyclobutyl, cyclopentyl, cyclohexyl, cycloheptyl or cyclooctyl;
C₃-C₈-cycloalkyl-C₁-C₆-alkyl: for example cyclopropylmethyl, cyclobutylmethyl, cyclopentylmethyl, cyclohexylmethyl, cycloheptylmethyl, cyclooctylmethyl, 2-(cyclopropyl)ethyl, 2-(cyclobutyl)ethyl, 2-(cyclopentyl)ethyl, 2-(cyclohexyl)ethyl, 2-(cycloheptyl)ethyl, 2-(cyclooctyl)ethyl, 3-(cyclopropyl)propyl, 3-(cyclobutyl)propyl, 3-(cyclopentyl)propyl, 3-(cyclohexyl)propyl, 3-(cycloheptyl)propyl, 3-(cyclooctyl)propyl, 4-(cyclopropyl)butyl, 4-(cyclobutyl)butyl, 4-(cyclopentyl)butyl, 4-(cyclohexyl)butyl, 4-(cycloheptyl)butyl, 4-(cyclooctyl)butyl, 5-(cyclopropyl)pentyl, 5-(cyclobutyl)pentyl, 5-(cyclopentyl)pentyl, 5-(cyclohexyl)pentyl, 5-(cycloheptyl)pentyl, 5-(cyclooctyl)pentyl, 6-(cyclopropyl)hexyl, 6-(cyclobutyl)hexyl, 6-(cyclopentyl)hexyl, 6-(cyclohexyl)hexyl, 6-(cycloheptyl)hexyl or 6-(cyclooctyl)hexyl;
C₃-C₈-cycloalkyloxy-C₁-C₄-alkyl: cyclopropyloxymethyl, 1-cyclopropyloxyethyl, 2-cyclopropyloxyethyl, 1-cyclopropyloxyprop-1-yl, 2-cyclopropyloxyprop-1-yl, 3-cyclopropyloxyprop-1-yl, 1-cyclopropyloxybut-1-yl, 2-cyclopropyloxybut-1-yl, 3-cyclopropyloxybut-1-yl, 4-cyclopropyloxybut-1-yl, 1-cyclopropyloxybut-2-yl, 2-cyclopropyloxybut-2-yl, 3-cyclopropyloxybut-2-yl, 4-cyclopropyloxybut-2-yl, 1-(cyclopropyloxymethyl)eth-1-yl, 1-(cyclopropyloxymethyl)-1-(CH₃)eth-1-yl, 1-(cyclopropylmethyloxy)prop-1-yl, cyclobutyloxymethyl, 1-cyclobutyloxyethyl, 2-cyclobutyloxyethyl, 1-cyclobutyloxyprop-1-yl, 2-cyclobutyloxyprop-1-yl, 3-cyclobutyloxyprop-1-yl, 1-cyclobutyloxybut-1-yl, 2-cyclobutyloxybut-1-yl, 3-cyclobutyloxybut-1-yl, 4-cyclobutyloxybut-1-yl, 1-cyclobutyloxybut-2-yl, 2-cyclobutyloxybut-2-yl, 3-cyclobutyloxybut-2-yl, 4-cyclobutyloxybut-2-yl, 1-(cyclobutyloxymethyl)eth-1-yl, 1-(cyclobutyloxymethyl)-1-(CH₃)eth-1-yl, 1-(cyclobutyloxymethyl)prop-1-yl, cyclopentyloxymethyl, 1-cyclopentyloxyethyl, 2-cyclopentyloxyethyl, 1-cyclopentyloxyprop-1-yl, 2-cyclopentyloxyprop-1-yl, 3-cyclopentyloxyprop-1-yl, 1-cyclopentyloxybut-1-yl, 2-cyclopentyloxybut-1-yl, 3-cyclopentyloxybut-1-yl, 4-cyclopentyloxybut-1-yl, 1-cyclopentyloxybut-2-yl, 2-cyclopentyloxybut-2-yl, 3-cyclopentyloxybut-2-yl, 4-cyclopentyloxybut-2-yl, 1-(cyclopentyloxymethyl)eth-1-yl, 1-(cyclopentyloxymethyl)-1-(CH₃)-eth-1-yl, 1-(cyclopentyloxymethyl)prop-1-yl, cyclohexyloxymethyl, 1-cyclohexyloxyethyl, 2-cyclohexyloxyethyl, 1-cyclohexyloxyprop-1-yl, 2-cyclohexyloxyprop-1-yl, 3-cyclohexyloxyprop-1-yl, 1-cyclohexyloxybut-1-yl, 2-cyclohexyloxybut-1-yl, 3-cyclohexyloxybut-1-yl, 4-cyclohexyloxybut-1-yl, 1-cyclohexyloxybut-2-yl, 2-cyclohexyloxybut-2-yl, 3-cyclohexyloxybut-2-yl, 4-cyclohexyloxybut-2-yl, 1-(cyclohexyloxymethyl)eth-1-yl, 1-(cyclohexyloxymethyl)-1-(CH₃)eth-1-yl, 1-(cyclohexyloxymethyl)prop-1-yl, cycloheptyloxymethyl, 1-cycloheptyloxyethyl, 2-cycloheptyloxyethyl, 1-cycloheptyloxyprop-1-yl, 2-cycloheptyloxyprop-1-yl, 3-cycloheptyloxyprop-1-yl, 1-cycloheptyloxybut-1-yl, 2-cycloheptyloxy-but-1-yl, 3-cycloheptyloxybut-1-yl, 4-cycloheptyloxybut-1-yl, 1-cycloheptyloxybut-2-yl, 2-cycloheptyloxybut-2-yl, 3-cycloheptyloxybut-2-yl, 4-cycloheptyloxybut-2-yl, 1-(cycloheptyloxymethyl)eth-1-yl, 1-(cycloheptyloxymethyl)-1-(CH₃)eth-1-yl, 1-(cycloheptyloxymethyl)prop-1-yl, cyclooctyloxymethyl, 1-cyclooctyloxyethyl, 2-cyclooctyloxyethyl, 1-cyclooctyloxyprop-1-yl, 2-cyclooctyloxyprop-1-yl, 3-cyclooctyloxyprop-1-yl, 1-cyclooctyloxybut-1-yl, 2-cyclooctyloxybut-1-yl, 3-cyclooctyloxybut-1-yl, 4-cyclooctyloxybut-1-yl, 1-cyclooctyloxybut-2-yl, 2-cyclooctyloxybut-2-yl, 3-cyclooctyloxybut-2-yl, 4-cyclooctyloxybut-2-yl, 1-(cyclooctyloxymethyl)eth-1-yl, 1-(cyclooctyloxymethyl)-1-(CH₃)eth-1-yl or 1-(cyclooctyloxymethyl)prop-1-yl, in particular C₃-C₆-cycloalkoxymethyl or 2-(C₃-C₆-cycloalkoxy)ethyl;
C₃-C₈-cycloalkyl which contains a carbonyl or a thiocarbonyl ring member: for example cyclobutanon-2-yl, cyclobutanon-3-yl, cyclopentanon-2-yl, cyclopentanon-3-yl, cyclohexanon-2-yl, cyclohexanon-4-yl, cycloheptanon-2-yl, cyclooctanon-2-yl, cyclobutanethion-2-yl, cyclobutanethion-3-yl, cyclopentanethion-2-yl, cyclopentanethion-3-yl, cyclohexanethion-2-yl, cyclohexanethion-4-yl, cycloheptanethion-2-yl or cyclooctanethion-2-yl, preferably cyclopentanon-2-yl or cyclohexanon-2-yl;
C₃-C₈-cycloalkyl in which one or two nonadjacent methylene groups are replaced by hetero atoms: oxiranyl, aziridinyl, 2- or 3-oxetanyl, 2- or 3-tetrahydrofuranyl, 2- or 3-tetrahydrothienyl, 1-, 2- or 3-pyrrolidinyl, 1-, 2-, 3- or 4-piperidinyl, 1- or 2-piperazinyl, 2-, 3- or 4-morpholinyl;
C₃-C₈-cycloalkyl in which one or two nonadjacent methylene groups are replaced by hetero atoms and which contain a carbonyl or thiocarbonyl group: 2-pyrrolidon-1-yl, 2-pyrrolidon-3-yl, -4-yl or -5-yl, 3-tetrahydrofuranon-2-yl, -4-yl or -5-yl, 2-tetrahydrofuranon-3-yl, -4-yl or -5-yl, tetrahydrothiophen-3-on-2-yl, -4-yl or -5-yl, tetrahydrothiophen-2-on-3-yl, -4-yl or -5-yl, 2-, morpholin-2-on-1-yl, -3-yl, -5-yl or -6-yl, caprolactam-1-yl, -3-yl, -4-yl, -5-yl, -6-yl or -7-yl.

Examples of mono- and bicyclic hetaryl having 5 to 10 ring members are monocycles such as furyl, for example 2-furyl and 3-furyl, thienyl, such as 2-thienyl and 3-thienyl, pyrrolyl, such as 2-pyrrolyl and 3-pyrrolyl, isoxazolyl, such as 3-isoxazolyl, 4-isoxazolyl and 5-isoxazolyl, isothiazolyl, such as 3-isothiazolyl, 4-isothiazolyl and 5-isothiazolyl, pyrazolyl, such as 3-pyrazolyl, 4-pyrazolyl and 5-pyrazolyl, oxazolyl, such as 2-oxazolyl, 4-oxazolyl and 5-oxazolyl, thiazolyl, such as 2-thiazolyl, 4-thiazolyl and 5-thiazolyl, imidazolyl, such as 2-imidazolyl and 4-imidazolyl, oxadiazolyl, such as 1,2,4-oxadiazol-3-yl, 1,2,4-oxadiazol-5-yl and 1,3,4-oxadiazol-2-yl, thiadiazolyl, such as 1,2,4-thiadiazol-3-yl, 1,2,4-thiadiazol-5-yl and 1,3,4-thiadiazol-2-yl, triazolyl, such as 1,2,4-triazol-1-yl, 1,2,4-triazol-3-yl and 1,2,4-triazol-4-yl, pyridinyl, such as 2-pyridinyl, 3-pyridinyl and 4-pyridinyl, pyridazinyl, such as 3-pyridazinyl and 4-pyridazinyl, pyrimidinyl, such as 2-pyrimidinyl, 4-pyrimidinyl and 5-pyrimidinyl, furthermore 2-pyrazinyl, 1,3,5-triazin-2-yl and 1,2,4-triazin-3-yl, and, as bicycles, the benzo-fused derivatives of the above-mentioned monocycles, such as quinolinyl, isoquinolinyl, indolyl, benzothienyl, benzofuranyl, benzoxazolyl, benzothiazolyl, benzoisothiazolyl, benzimidazolyl, benzopyrazolyl, benzothiadiazolyl, benzotriazolyl, in particular pyridyl, pyrimidyl, furanyl and thienyl.

With respect to the herbicidal action of the compounds according to the invention, the variables have, independently of one another, preferably in combination with one another, the following meanings:
n: 1, 2 or 3;
X and X¹: independently of one another CO;
Ar² and Ar^{ω}: independently of one another phenyl, thienyl, furanyl, pyrrolyl, pyridyl, pyrimidinyl, naphthyl or quinolinyl, which is unsubstituted or substituted in the manner described above and which is preferably unsubstituted or carries 1, 2 or 3 substituents selected from the group consisting of halogen, hydroxyl, mercapto, nitro, cyano, CO₂H, HC(O), HC(O)O, C₁-C₈-alkyl, C₂-C₈-alkenyl, C₂-C₈-alkynyl, C₁-C₈-alkoxy, C₂-C₈-alkenyloxy, C₃-C₈-alkynyloxy, C₁-C₈-alkylthio, C₁-C₈-alkylsulfinyl, C₁-C₈-alkylsulfonyl, C₁-C₈-alkylcarbonyl, C₁-C₈-alkylcarbonyloxy, C₁-C₈-alkyloxycarbonyl, C₅-C₈-cycloalkyloxycarbonyl, C₁-C₈-haloalkyl, C₁-C₈-haloalkoxy, C₂-C₈-haloalkenyloxy, C₁-C₈-haloalkylthio, C₁-C₈-haloalkylsulfonyl, NH₂, NH—C₁-C₄-alkyl, N(C₁-C₄-alkyl)₂, where two substituents located on adjacent carbons may also form a C₃-C₅-alkylene chain in which one or two nonadjacent methylene groups are replaced by oxygen atoms and which may carry, as substituents, 1, 2, 3 or 4 fluorine atoms and/or 1 or 2 methyl groups, for example 1,3-propylene, 1,4-butylene, 1,5-pentylene, 1,3-dioxapropylene, 1,4-dioxabutylene or 2,2-difluoro-1,3-dioxapropylene. Among the radicals Ar² and Ar^{ω}, preference is given to those having, in the position ortho to the atom attached to the group X or X¹, a substituent or a fused ring;
R¹: hydrogen, C₁-C₁₂-alkyl, C₂-C₁₂-alkenyl, C₃-C₈-alkynyl, hydroxyl-C₁-C₄-alkyl, amino-C₁-C₄-alkyl, C₁-C₆-alkoxy-C₁-C₄-alkyl, C₁-C₆-alkoxy-C₁-C₄-alkoxy-C₁-C₄-alkyl, C₁-C₆-alkylamino-C₁-C₄-alkyl, di-(C₁-C₆-alkyl)amino-C₁-C₄-alkyl, C₂-C₆-alkenyloxy-C₁-C₄-alkyl, C₃-C₄-alkynyloxy-C₁-C₄-alkyl, C₁-C₆-alkylthio-C₁-C₄-alkyl, C₂-C₆-alkenylthio-C₁-C₄-alkyl, where the 8 groups mentioned above may also carry 1, 2, 3, 4 or 5 halogen atoms selected from the group consisting of fluorine and chlorine,
C₃-C₈-cycloalkyl, C₃-C₈-cycloalkyl-C₁-C₄-alkyl or C₅-C₆-cycloalkenyl, where the cycles of the three last-mentioned groups may, instead of a methylene group, have an oxygen or sulfur atom, may carry a carbonyl group and may carry one to four substituents selected from the group consisting of fluorine, chlorine, C₁-C₄-alkyl and C₁-C₄-alkoxy,
phenyl-C₁-C₄-alkyl, 2-, 3- or 4-pyridyl-C₁-C₄-alkyl, 2- or 3-thienyl-C₁-C₄-alkyl, which may have 1, 2, 3 or 4 substituents selected from the group consisting of halogen, C₁-C₄-alkyl, C₁-C₄-alkoxy, C₁-C₄-haloalkyl, C₁-C₄-haloalkoxy, cyano and nitro;
R² and R³: independently of one another preferably hydrogen, C₁-C₁₂-alkyl, C₂-C₁₂-alkenyl, C₃-C₈-alkynyl, hydroxyl-C₁-C₄-alkyl, amino-C₁-C₄-alkyl, C₁-C₆-alkoxy-C₁-C₄-alkyl, C₁-C₆-alkoxy-C₁-C₄-alkoxy-C₁-C₄-alkyl, C₁-C₆-alkylamino-C₁-C₄-alkyl, di-(C₁-C₆-alkyl)amino-C₁-C₄-alkyl, C₂-C₆-alkenyloxy-C₁-C₄-alkyl, C₃-C₄-alkynyloxy-C₁-C₄-alkyl, C₁-C₆-alkylthio-C₁-C₄-alkyl, C₂-C₆-alkenylthio-C₁-C₄-alkyl, where the 8 groups mentioned above may also carry 1, 2, 3, 4 or 5 halogen atoms selected from the group consisting of fluorine and chlorine,
C₃-C₈-cycloalkyl, C₃-C₈-cycloalkyl-C₁-C₄-alkyl or C₅-C₆-cycloalkenyl, where the cycles of the three last-mentioned groups may, instead of a methylene group, have an oxygen atom, a sulfur atom or an NH group, may have a carbonyl group and may carry one to four substituents selected from the group consisting of fluorine, chlorine, C₁-C₄-alkyl and C₁-C₄-alkoxy,
phenyl, 2- or 3-thienyl, 2-, 3- or 4-pyridyl, 2-, 4- or 5-thiazolyl, 2-, 4- or 5-pyrimidinyl, 3- or 4-pyridazinyl, 2-benzothiazolyl, phenyl-C₁-C₄-alkyl, phenoxy-C₁-C₄-alkyl, 2-, 3- or 4-pyridyl-C₁-C₄-alkyl, 2- or 3-thienyl-C₁-C₄-alkyl, where the aromatic or heteroaromatic groups mentioned above may have 1, 2, 3 or 4 substituents selected from the group consisting of halogen, C₁-C₄-alkyl, C₁-C₄-alkoxy, C₁-C₄-haloalkyl, C₁-C₄-haloalkoxy, cyano and nitro, where R³ is in particular hydrogen or C₁-C₄-alkyl, or
R² and R³ together with the nitrogen atom to which they are attached form a saturated 5-, 6- or 7-membered nitrogen heterocycle which may have 1, 2, 3 or 4 substituents selected from the group consisting of C₁-C₄-alkyl and C₁-C₄-alkoxy and in which one methylene group may be replaced by an oxygen atom, a sulfur atom, an NH or a C₁-C₄-alkylimino group, for example an azetidine, pyrrolidine, pyrroline, piperidine, morpholine or N⁴-methyl or N⁴-ethylpiperazine radical.

Ar² and Ar^{ω} are in particular, independently of one another, phenyl, 2- or 3-thienyl, 2- or 3-furanyl, 2-, 3- or 4-pyridyl, 2-, 4- or 5-pyrimidyl, 2-, 3-, 5- or 8-quinolinyl or 1- or 2-naphthyl, in particular phenyl, 2- or 3-thienyl, 3-pyridyl or 8-quinolinyl, which is substituted in the manner described above and may preferably have 1, 2 or 3 substituents selected from the group consisting of fluorine, chlorine, bromine, iodine, hydroxyl, C₁-C₆-alkyl, allyl, methallyl, C₁-C₆-alkoxy, allyloxy, methallyloxy, 2-butenyloxy, propargyloxy, methylthio, ethylthio, methylsulfinyl, ethylsulfinyl, methylsulfonyl, ethylsulfonyl, trifluoromethyl, difluoromethoxy, trifluoromethoxy, methylenedioxy, difluoromethylenedioxy, difluoromethylthio, trifluoromethylthio, trifluoromethylsulfinyl, trifluoromethylsulfonyl, hydroxyl, acetoxy, mercapto, nitro, cyano, —COOCH₃, —COOC₂H₅, —COOC₃H₇, amino and dimethylamino. The substituents are selected in particular from the group consisting of fluorine, chlorine, bromine, iodine, trifluoromethyl, hydroxyl, acetoxy, C₁-C₆-alkyl and C₁-C₄-alkoxy.

Examples of particularly preferred groups Ar^{ω} are phenyl, 2-methylphenyl, 1-naphthyl, 2-fluorophenyl, 2-chlorophenyl, 2-bromophenyl, 2-chloro-3-pyridyl, 2-methylphenyl, 2-methoxyphenyl, 2,3-, 2,4-, 2,5- and 2,6-difluoromethylphenyl, 2,3- and 2,4-dimethylphenyl, 2,3- and 2,4-dimethoxyphenyl, 2-furanyl, 2-thienyl, 2-fluorothiophen-3-yl and 3-fluorothiophen-2-yl.

Examples of particularly preferred groups Ar² are phenyl, 2-methylphenyl, 1-naphthyl, 2-fluorophenyl, 2-chlorophenyl, 2-bromophenyl, 2-iodophenyl, 2-chloro-3-pyridyl, 2-methylphenyl, 2-methoxyphenyl, 2-acetoxyphenyl, 2-trifluoromethylphenyl, 2-nitrophenyl, 2-aminophenyl, 2,3-, 2,4-, 2,5- and 2,6-difluoromethylphenyl, 2,3- and 2,4-dimethylphenyl, 2,3- and 2,4-dimethoxyphenyl, 2,3,4- and 2,4,5-trifluorophenyl, 2,3,4-trichlorophenyl, 2,4,6- and 2,3,6-trimethylphenyl, 2,3,6-trimethyl-4-methoxyphenyl, 2-chloro-4-fluorophenyl, 2-amino-4-chlorophenyl, 2-amino-5-chlorophenyl, 2-amino-4-hydroxyphenyl, 2-amino-3,5-dichlorophenyl, 3,5-dichloro-2-hydroxyphenyl, 2-furanyl, 2-thienyl, 2,5-dichlorothiophen-3-yl, 2-fluorothiophen-3-yl, 3-fluorothiophen-2-yl, 1-naphthyl, 8-quinolinyl. Also preferred are 3-chlorophenyl, 4-chlorophenyl, 4-(n-butyl)phenyl, 4-(n-hexyl)phenyl and 4-(n-heptyl)phenyl.

R¹ is in particular:
C₁-C₈-alkyl, such as methyl, ethyl, n- or isopropyl, n-, s-, i- or t-butyl, n-pentyl, 3-methylbutyl, 1,1-dimethylpropyl, 2,2-dimethylpropyl, n-hexyl, 3,3-dimethylbutyl, 2-ethylhexyl, n-heptyl, n-octyl,
unsubstituted or C₁-C₄-alkyl-, halogen- or C₁-C₄-alkoxy-substituted C₃-C₆-cycloalklyl, such as cyclopropyl, cyclobutyl, cyclopentyl, cyclohexyl, 2-, 3- and 4-methylcyclohexyl,
C₃-C₆-cycloalkyl, in which one CH₂ group is replaced by oxygen, sulfur, NH or methylimino and which may have a carbonyl group, such as 2,2,6,6-tetramethylpiperidin-4-yl, 2-oxotetrahydrofuran-3-yl, 3-oxotetrahydrothiophen-3-yl,
C₃-C₆-cycloalkyl-C₁-C₂-alkyl, such as cyclopropylmethyl, cyclobutylmethyl, cyclopentylmethyl, cyclopentylethyl, cyclohexylmethyl, cyclohexylethyl,
C₅-C₆-cycloalkenyl, such as 2- and 3-cyclopentenyl,
C₁-C₆-haloalkyl, such as 2-chloro-, 2-bromo- or 2-iodoethyl, 2,2,2-trichloro- or 2,2,2-trifluoroethyl, 3-chloro- or 3-bromopropyl, 4-chloro- or 4-bromobutyl, 5-chloro- or 5-bromopentyl, 6-chloro- or 6-bromohexyl,
C₃-C₆-alkenyl or C₃-C₆-haloalkenyl, such as allyl and methallyl, 3-chloroallyl, 2-chloro- or 2-bromoallyl, 2,3-dichloroallyl,
C₁-C₄-alkoxy-C₂-C₃-alkyl or C₁-C₄-alkoxy-C₂-C₃-alkoxy-C₂-C₃-alkyl, such as 2-methoxyethyl, 2-ethoxyethyl, 2-propoxyethyl, 2-(n-, iso-, s- and t-butoxy)ethyl, 2-(2-methoxyethoxy)ethyl, 2-(2-ethoxyethoxy)ethyl, 3-(2-methoxyethoxy)propyl,
C₁-C₄-alkylthio-C₂-C₃-alkyl, such as 2-methylthioethyl, 2-ethylthioethyl, 2-propylthioethyl,
di-(C₁-C₄-alkyl)amino-C₂-C₃-alkyl, such as 2-dimethylaminoethyl, 2-diethylaminoethyl, 3-dimethylaminopropyl, 3-diethylaminopropyl,
phenyl-C₁-C₂-alkyl, phenoxy-C₁-C₂-alkyl, which may be substituted by halogen, alkyl or alkylthio, such as benzyl, phenylethyl, 2-, 3- or 4-fluoro-, 2-, 3- or 4-chloro- or 2-, 3- or 4-bromobenzyl, 2-, 3- or 4-fluoro-, 2-, 3- or 4-chloro- or 2-, 3- or 4-bromophenylethyl, 2-, 3- or 4-methoxy-, 2-, 3- or 4-methylthiobenzyl or 2-, 3- or 4-methoxy-, 2-, 3- or 4-methylthiophenylethyl, 2-, 3- or 4-methyl-, 2-, 3- or 4-ethyl-, 2-, 3- or 4-(n- or i-propyl)benzyl, 2-, 3- or 4-methyl-, 2-, 3- or 4-ethyl-, 2-, 3- or 4-(n- or i-propyl)phenylethyl, 2-, 3- or 4-trifluoromethylbenzyl, 2-, 3- or 4-trifluorophenylethyl, thien-2- or -3-ylmethyl or pyridin-2-, -3- or -4-ylmethyl, 2-phenoxyethyl, 2-(2-, 3- or 4-fluoro, -chloro- or -bromophenoxy)ethyl.

R² is in particular:
C₁-C₈-alkyl,
unsubstituted or C₁-C₄-alkyl-, halogen- or C₁-C₄-alkoxy-substituted C₃-C₆-cycloalkyl,
C₃-C₆-cycloalkyl in which one CH₂ group is replaced by oxygen, sulfur, NH or methylimino and which may have a carbonyl group,
C₃-C₆-cycloalkyl-C₁-C₂-alkyl,
C₅-C₆-cycloalkenyl,
C₁-C₆-haloalkyl,
C₃-C₆-alkenyl,
C₃-C₆-haloalkenyl,
C₁-C₄-alkoxy-C₂-C₃-alkyl,
C₁-C₄-alkoxy-C₂-C₃-alkoxy-C₂-C₃-alkyl,
C₁-C₄-alkylthio-C₂-C₃-alkyl,
di-(C₁-C₄-alkyl)amino-C₂-C₃-alkyl,
phenyl, thienyl, furanyl, pyridyl, pyrimidyl, naphthyl, phenyl-C₁-C₂-alkyl, phenoxy-C₁-C₂-alkyl, 2- or 3-thienyl-C₁-C₂-alkyl, 2- or 3-pyridyl-C₁-C₂-alkyl, where the 12 last-mentioned groups may carry 1, 2, 3 or 4 substituents selected from the group consisting of fluorine, chlorine, bromine, iodine, and/or 1 or 2 substituents selected from the group consisting of C₁-C₆-alkyl, C₁-C₆-alkoxy, allyloxy, methallyloxy, 2-butenyloxy, propargyloxy, methylthio, ethylthio, methylsulfinyl, ethylsulfinyl, methylsulfonyl, ethylsulfonyl, trifluoromethyl, difluoromethoxy, trifluoromethoxy, methylenedioxy, difluoromethylenedioxy, difluoromethylthio, trifluoromethylthio, trifluoromethylsulfinyl, trifluoromethylsulfonyl, hydroxyl, acetoxy, mercapto, nitro, cyano, —COOCH₃, —COOC₂H₅, —COOC₃H₇, amino or dimethylamino.

R³ is in particular hydrogen, methyl or ethyl or, together with R², forms an azetidine, pyrrolidine, pyrroline, piperidine, morpholine or N⁴-methyl- or N⁴-ethylpiperazine radical.

To prepare the 2,ω-diaminocarboxylic acid compounds of the formula I according to the invention, in general, a compound of the formula II, 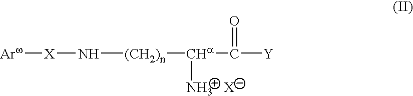
in which Ar^{ω}, X, n and Y are as defined above and X^{⊖} is a monovalent anion or an anion equivalent, for example Cl^{⊖}, Br^{⊖} or ½ SO₄^{2⊖} of a mineral acid, for example Cl^{⊖}, Br^{⊖} or ½ SO₄^{2⊖}, is reacted with an aromatic acyl halide of the formula III
Ar²-X¹-Hal (III)
in which Ar² and X¹ are as defined above and Hal is chlorine, bromine or iodine. The compounds II in which Y≠OH and Ar^{ω} is different from unsubstituted phenyl are novel and also form part of the subject matter of the present invention. The aromatic acyl halides III are known, and some of them are commercially available, or they can be prepared by known processes.

The reaction of the compound II with the compound III is preferably carried out in the presence of a base. The base serves to neutralize the mineral acid H-Hal and H—X formed during the reaction. Suitable bases are all inorganic or organic bases which are customarily used for acylations, for example alkali metal hydroxides, such as NaOH or KOH, alkali metal carbonates, such as Na₂CO₃ or K₂CO₃, alkali metal bicarbonates, such as NaHCO₃, tertiary amines, such as triethylamine, N-methylpiperidine, N-ethyldiisopropylamine, N,N-dimethylaminopyridine, pyridine, diazabicyclooctane (DABCO), diazabicyclononene (DBN) or diazabicycloundecene (DBU). The base is preferably employed in at least equimolar quantity, in particular in a quantity of from 1 to 3 mol per mole of acid H-Hal and H—X to be neutralized.

The reaction of the compound II with the compound III is preferably carried out in a solvent or diluent. Suitable solvents/diluents are water, diethyl ether, tetrahydrofuran, acetonitrile, ethyl acetate, dichloromethane or toluene.

The reaction temperatures can be varied within a certain range, which is defined by the stability of the acyl chloride III. The reaction is preferably carried out at temperatures in the range from 0 to 30° C.

Work-up is carried out by customary methods, for example by admixing the reaction mixture with cold water, separating off the organic phase and, after drying, concentrating it under reduced pressure. The residue that remains can, if required, be freed of any impurities that may be present in a customary manner by chromatography or crystallization.

The compounds of the formula II can be prepared by initially reacting, in a first step, a partially protected 2,ω-diaminocarboxylic acid of the formula IV or its acid addition salt 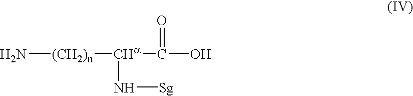
in which n is as defined above and Sg is a protective group with an acyl halide of the formula V
Ar^{ω}-X-Hal (V)
in which Ar^{ω} and X are as defined above and Hal is chlorine, bromine or iodine, reacting, in a second step, the resulting compound of the formula VI 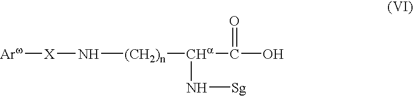
with an alcohol of the formula R¹—OH or an amine of the formula R²R³NH in the presence of a suitable condensing agent and finally removing the protective group Sg.

Suitable protective groups are those which can be removed under conditions which do not result in a cleavage of the NH—X bond in the compounds of the formula VI. Suitable protective groups are known from peptide chemistry. They include, in particular, protective groups which are removed by action of acids which preferably have an acidity above that of acetic acid, for example the tert-butoxycarbonyl group, the 1-adamantyloxycarbonyl group and the 2-(trimethylsilyl)ethoxycarbonyl group.

The reaction of the compound VI with an alcohol HOR¹ is carried out using the customary methods for esterifying carboxylic acids in the presence of esterification catalysts and/or customary dehydrating agents as condensing agents or by reaction in the presence of esterification catalysts with removal of the water of reaction formed during the reaction. The esterification catalyst used is preferably a hydrogen chloride donor, such as trimethylchlorosilane or thionyl chloride. If the protective group is chosen appropriately, both the esterification and the removal of the protective group on the α-amino group take place simultaneously. Such reactions are described in the prior art, for example by E. J. Corey et al., Tetrahedron Lett. 33 (1992), 6807; Bang-Chi Chen et al., J. Org. Chem. 64 (1999), 9294; Z.-Y. Chang et al., J. Org. Chem. 55 (1990), 3475, which are included herein by way of reference.

Suitable condensing agents for the reaction of compound VI with the amine HNR₂R₃ are all reagents which can activate free carboxyl groups, such as: propanephosphonic anhydride (PPPA, H. Wissmann et al., Angew. Chem. 92 (1980), 129; H. Wissmann, Phosphorus, Sulfur 30 (1986), 645; M. Feigel, J. Am. Chem. Soc 108 (1986), 181), N-ethoxycarbonyl-2-ethoxy-1,2-dihydroquinoline (EEDQ, B. Belleau et al., J. Am. Chem. Soc. 90 (1968), 1651), diphenylphosphoryl azide (DPPA, Shun-ichi-Yamada et al., J. Am. Chem. Soc. 94 (1972), 6203) and diethylphosphoryl cyanide (DEPC, Shun-ichi-Yamada et al., Tetrahedron Lett. 18 (1973), 1595), carbodiimides (Houben-Weyl, Methoden der Organischen Chemie [Methods of Organic Chemistry], Vol. 15/2, pages 103-115, 4th Edition, G. Thieme Verlag), to mention but a few condensing agents by way of example. The reaction conditions described in the references can be applied to the reaction according to the invention of the compound VI with the amine HNR₂R₃, and these publications are likewise included herein by way of reference.

The removal of the protective group Sg, for example of the tert-butoxycarbonyl (BOC) group from the resulting compounds VII 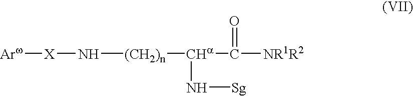
in which Ar^{ω}, X, n, R¹, R² and Sg are as defined above is generally carried out using an acid, preferably with the aid of trifluoroacetic acid, for example by the methods described by B. Lundt et al., Int. J. Pept. Protein Res., 12 (1978), 258), or, for example, using 2N hydrogen chloride in dioxane, according to the methods described by R. Andruszkievicz et al., J. Med. Chem. 30 (1987), 1715) and gives, in good yields, the above-mentioned intermediates of the formula (II) according to the invention.

The carboxylic and sulfonic halides Ar^{ω}-X-Hal and Ar²-X¹-Hal used as starting materials in these reactions are known or can be prepared by known methods.

The 2-N-protected 2,ω-diamino acids IV are likewise known, commercially available or they can be prepared by known methods, for example according to: Houben-Weyl, Methoden der organischen Chemie, Vol. 15/1, G. Thieme Verlag, (compounds IV, n=3 and 4); N. Kucharczyk et al., Synth. Commun. 19 (1989), 1603; M. Waki et al., Synthesis, 266 (1981) and Lin-Hua Zhang et al., J. Org. Chem. 62 (1997), 6918 (compounds VI, n=1 and 2).

The compounds of the formula I and their agriculturally useful salts are suitable, both in the form of racemates, enantiomer mixtures and in the form of the pure enantiomers, as herbicides. The herbicidal compositions comprising the compounds I control vegetation on non-crop areas very efficiently, especially at high rates of application. They act against broad-leaved weeds and harmful grasses in crops such as wheat, rice, maize, soya and cotton without causing any significant damage to the crop plants. This effect is mainly observed at low rates of application.

Depending on the application method used, the compounds I or the compositions comprising them can additionally be employed in a further number of crop plants for eliminating undesirable plants. Examples of suitable crops are the following:

Allium cepa, Ananas comosus, Arachis hypogaea, Asparagus officinalis, Beta vulgaris spec. altissima, Beta vulgaris spec. rapa, Brassica napus var. napus, Brassica napus var. napobrassica, Brassica rapa var. silvestris, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N. rustica), Olea europaea, Oryza sativa, Phaseolus lunatus, Phaseolus vulgaris, Picea abies, Pinus spec., Pisum sativum, Prunus avium, Prunus persica, Pyrus communis, Ribes sylvestre, Ricinus communis, Saccharum officinarum, Secale cereale, Solanum tuberosum, Sorghum bicolor (S. vulgare), Theobroma cacao, Trifolium pratense, Triticum aestivum, Triticum durum, Vicia faba, Vitis vinifera and Zea mays.

In addition, the compounds I may also be used in crops which tolerate the action of herbicides owing to breeding, including genetic engineering methods.

The compounds I, or the compositions comprising them, can be used for example in the form of ready-to-spray aqueous solutions, powders, suspensions, also highly-concentrated aqueous, oily or other suspensions or dispersions, emulsions, oil dispersions, pastes, dusts, materials for broadcasting or granules, by means of spraying, atomizing, dusting, broadcasting, watering or by treating the seed or mixing with the seed. The use forms depend on the intended aims; in any case, they should ensure the finest possible distribution of the active compounds according to the invention. The herbicidal compositions comprise a herbicidally effective amount of at least one compound of the formula I or an agriculturally useful salt of I and auxiliaries customarily used for formulating crop protection agents.

Essentially, suitable inert auxiliaries include: mineral oil fractions of medium to high boiling point, such as kerosene and diesel oil, furthermore coal tar oils and oils of vegetable or animal origin, aliphatic, cyclic and aromatic hydrocarbons, e.g. paraffin, tetrahydronaphthalene, alkylated naphthalenes and their derivatives, alkylated benzenes and their derivatives, alcohols such as methanol, ethanol, propanol, butanol and cyclohexanol, ketones such as cyclohexanone, strongly polar solvents, e.g. amines such as N-methylpyrrolidone and water.

Aqueous use forms can be prepared from emulsion concentrates, suspensions, pastes, wettable powders or water-dispersible granules by adding water. To prepare emulsions, pastes or oil dispersions, the compounds I, either as such or dissolved in an oil or solvent, can be homogenized in water by means of a wetting agent, tackifier, dispersant or emulsifier. Alternatively, it is possible to prepare concentrates consisting of active substance, wetting agent, tackifier, dispersant or emulsifier and, if desired, solvent or oil, which are suitable for dilution with water.

Suitable surfactants are the alkali metal salts, alkaline earth metal salts and ammonium salts of aromatic sulfonic acids, e.g. ligno-, phenol-, naphthalene- and dibutylnaphthalenesulfonic acid, and of fatty acids, alkyl- and alkylarylsulfonates, alkyl sulfates, lauryl ether sulfates and fatty alcohol sulfates, and salts of sulfated hexa-, hepta- and octadecanols, and also of fatty alcohol glycol ethers, condensates of sulfonated naphthalene and its derivatives with formaldehyde, condensates of naphthalene, or of the naphthalenesulfonic acids with phenol and formaldehyde, polyoxyethylene octylphenol ether, ethoxylated isooctyl-, octyl- or nonylphenol, alkylphenyl or tributylphenyl polyglycol ether, alkylaryl polyether alcohols, isotridecyl alcohol, fatty alcohol ethylene oxide condensates, ethoxylated castor oil, polyoxyethylene alkyl ethers or polyoxypropylene alkyl ethers, lauryl alcohol polyglycol ether acetate, sorbitol esters, lignosulfite waste liquors or methylcellulose.

Powders, materials for broadcasting and dusts can be prepared by mixing or grinding the active substances together with a solid carrier.

Granules, e.g. coated granules, impregnated granules and homogeneous granules, can be prepared by binding the active compounds to solid carriers. Solid carriers are mineral earths, such as silicas, silica gels, silicates, talc, kaolin, limestone, lime, chalk, bole, loess, clay, dolomite, diatomaceous earth, calcium sulfate, magnesium sulfate, magnesium oxide, ground synthetic materials, fertilizers such as ammonium sulfate, ammonium phosphate, ammonium nitrate and ureas, and products of vegetable origin, such as cereal meal, tree bark meal, wood meal and nutshell meal, cellulose powders, or other solid carriers.

The concentrations of the active compounds I in the ready-to-use preparations can be varied within wide ranges. In general, the formulations comprise from about 0.001 to 98% by weight, preferably 0.01 to 95% by weight, of at least one active compound. The active compounds are employed in a purity of from 90% to 100%, preferably from 95% to 100% (according to the NMR spectrum).

The compounds according to the invention can be formulated, for example, as follows:

I 20 parts by weight of the compound of Example 71 (see Table 6) are dissolved in a mixture consisting of 80 parts by weight of alkylated benzene, 10 parts by weight of the adduct of 8 to 10 mol of ethylene oxide to 1 mol of oleic acid N-monoethanolamide, 5 parts by weight of calcium dodecylbenzenesulfonate and 5 parts by weight of the adduct of 40 mol of ethylene oxide to 1 mol of castor oil. Pouring the solution into 100,000 parts by weight of water and finely distributing it therein gives an aqueous dispersion which comprises 0.02% by weight of the active compound.

II 20 parts by weight of the compound of Example 71 are dissolved in a mixture consisting of 40 parts by weight of cyclohexanone, 30 parts by weight of isobutanol, 20 parts by weight of the adduct of 7 mol of ethylene oxide to 1 mol of isooctylphenol and 10 parts by weight of the adduct of 40 mol of ethylene oxide to 1 mol of castor oil. Pouring the solution into 100,000 parts by weight of water and finely distributing it therein gives an aqueous dispersion which comprises 0.02% by weight of the active compound.

III 20 parts by weight of the active compound of Example 71 are dissolved in a mixture consisting of 25 parts by weight of cyclohexanone, 65 parts by weight of a mineral oil fraction of boiling point 210 to 280° C. and 10 parts by weight of the adduct of 40 mol of ethylene oxide to 1 mol of castor oil. Pouring the solution into 100,000 parts by weight of water and finely distributing it therein gives an aqueous dispersion which comprises 0.02% by weight of the active compound.

IV 20 parts by weight of the active compound of Example 71 are mixed thoroughly with 3 parts by weight of sodium diisobutylnaphthalenesulfonate, 17 parts by weight of the sodium salt of lignosulfonic acid from a sulfite waste liquor and 60 parts by weight of pulverulent silica gel, and the mixture is ground in a hammer mill. Finely distributing the mixture in 20,000 parts by weight of water gives a spray mixture which comprises 0.1% by weight of the active compound.

V 3 parts by weight of the active compound of Example 71 are mixed with 97 parts by weight of finely divided kaolin. This gives a dust which comprises 3% by weight of the active compound.

VI 20 parts by weight of the active compound of Example 71 are mixed intimately with 2 parts by weight of calcium dodecylbenzenesulfonate, 8 parts by weight of fatty alcohol polyglycol ether, 2 parts by weight of the sodium salt of a phenol/urea/formaldehyde condensate and 68 parts by weight of a paraffinic mineral oil. This gives a stable oily dispersion.

VII 1 part by weight of the compound of Example 71 is dissolved in a mixture consisting of 70 parts by weight of cyclohexanone, 20 parts by weight of ethoxylated isooctylphenol and 10 parts by weight of ethoxylated castor oil. This gives a stable emulsion concentrate.

VII 1 part by weight of the compound of Example 71 is dissolved in a mixture consisting of 80 parts by weight of cyclohexanone and 20 parts by weight of Wettol® EM 31 (=nonionic emulsifier based on ethoxylated castor oil). This gives a stable emulsion concentrate.

The herbicidal compositions or the active compounds can be applied pre- or post-emergence or together with the seeds of a crop plant. It is also possible to apply the herbicidal compositions or active compounds by sowing seed of a crop plant where the seeds have been pre-treated with the herbicidal compositions on active compounds. If the active compounds are less well tolerated by certain crop plants, application techniques may be used in which the herbicidal compositions are sprayed, with the aid of the spraying equipment, in such a way that they come into contact as little as possible, if at all, with the leaves of the sensitive crop plants, while the active compounds reach the leaves of undesirable plants growing underneath, or the bare soil surface (post-directed, lay-by).

The application rates of the active compound are from 0.001 to 10.0, preferably from 0.01 to 5.0 kg/ha of active substance (a.s.), depending on the control target, the season, the target plants and the growth stage.

To widen the activity spectrum and to achieve synergistic effects, the compounds of the formula I may be mixed with a large number of representatives of other herbicidal or growth-regulating active compound groups and then applied concomitantly. Suitable components for mixtures are, for example, 1,2,4-thiadiazoles, 1,3,4-thiadiazoles, amides, aminophosphoric acid and its derivatives, aminotriazoles, anilides, (het)aryloxyalkanoic acid and its derivatives, benzoic acid and its derivatives, benzothiadiazinones, 2-(hetaroyl/aroyl)-1,3-cyclohexandiones, hetaryl-aryl ketones, benzylisoxazolidinones, meta-CF₃-phenyl derivatives, carbamates, quinolinecarboxylic acid and its derivatives, chloroacetanilides, cyclohexenone oxime ether derivatives, diazines, dichloropropionic acid and its derivatives, dihydrobenzofurans, dihydrofuran-3-ones, dinitroanilines, dinitrophenols, diphenyl ether, dipyridyls, halocarboxylic acids and their derivatives, ureas, 3-phenyluracils, imidazoles, imidazolinones, N-phenyl-3,4,5,6-tetrahydrophthalimides, oxadiazoles, oxiranes, phenols, aryloxy- and hetaryloxyphenoxypropionic esters, phenylacetic acid and its derivatives, phenylpropionic acid and its derivatives, pyrazoles, phenylpyrazoles, pyridazines, pyridinecarboxylic acid and its derivatives, pyrimidyl ethers, sulfonamides, sulfonylureas, triazines, triazinones, triazolinones, triazolecarboxamides and uracils.

It may furthermore be advantageous to apply the compounds of the formula I alone or else concomitantly in combination with other herbicides, or in the form of a mixture with other crop protection agents, for example together with agents for controlling pests or phytopathogenic fungi or bacteria. Also of interest is the miscibility with mineral salt solutions, which are employed for treating nutritional and trace element deficiencies. Non-phytotoxic oils and oil concentrates may also be added.

The examples below are meant to illustrate the invention without limiting it.

### PREPARATION EXAMPLES

I Intermediates of the Formula VI

### Intermediate VI-1: (S)-N²-(tert-butoxycarbonyl)-N³-benzoyl-2,3-diaminopropionic Acid

At 5° C., 21.1 g (0.15 mol) of benzoyl chloride and 85 ml (0.17 mol) of aqueous 2N sodium hydroxide solution were added simultaneously from two dropping funnels, with stirring, to a solution of 30.6 g of (S)-N²-(tert-butoxycarbonyl)-2,3-diaminopropionic acid (0.15 mol) in 75 ml of 2N aqueous solution hydroxide solution (0.15 mol). The mixture was stirred at 25° C. for two hours and then extracted twice with in each case 100 ml of diethyl ether, acidified to pH 2.5 with 1N HCl solution and extracted three times with in each case 150 ml of ethyl acetate. The combined ethyl acetate extracts were washed successively with 1% strength NaHCO₃ solution and with water, dried over Na₂SO₄ and concentrated under reduced pressure. The semisolid residue was stirred with 100 ml of n-pentane and, after 14 hours, filtered off with suction and washed with n-pentane. This gave 36 g (83.9% of theory) of (S)-N²-(tert-butoxycarbonyl)-N³-benzoyl-2,3-diaminopropionic acid as fine white crystals of melting point 110-111° C.

The intermediates VI-2 to VI-34 listed in Table 1 were prepared similarly to the preparation of intermediate VI-1.

TABLE 1
Intermediates of the formula (VI) where Sg =
butoxycarbonyl

| | |
|---|---|
| | (VI) |
| 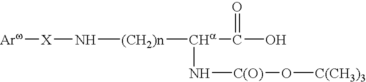 | |

| Intermediate | | | | Config. | |
|---|---|---|---|---|---|
| VI-No. | Ar^{ω} | X | n | C-α¹ | m.p. ° C. |
| 2 | C₆H₅ | CO | 1 | R | |
| 3 | C₆H₅ | CO | 1 | rac. | |
| 4 | 2-CH₃—C₆H₄² | CO | 1 | S | 137-138 |
| 5 | 2-CH₃—C₆H₄ | CO | 1 | R | |
| 6 | 1-C₁₀H₇³ | CO | 1 | S | 139-140 |
| 7 | 2-FC₆H₄ | CO | 1 | S | 119-121 |
| 8 | 2-FC₆H₄ | CO | 1 | R | |
| 9 | 2-FC₆H₄ | CO | 1 | rac. | |
| 10 | 2-FC₆H₄ | CO | 2 | S | 130-131 |
| 11 | 2-FC₆H₄ | CO | 2 | R | |
| 12 | 2-FC₆H₄ | CO | 2 | rac. | |
| 13 | 2-FC₆H₄ | CO | 4 | S | 89-91 |
| 14 | 2-Cl C₆H₄ | CO | 1 | S | 143-144 |
| 15 | 2-Cl C₆H₄ | CO | 1 | R | |
| 16 | 2-chloro-3-pyridyl | CO | 1 | S | 156-157 |
| 17 | 2-BrC₆H₄ | CO | 1 | S | 148-149 |
| 18 | 2,6-F₂C₆H₃ | CO | 1 | S | 134-136 |
| 19 | 2,3-F₂C₆H₃ | CO | 1 | S | 154-155 |
| 20 | 2,3-F₂C₆H₃ | CO | 1 | R | |
| 21 | 2,4-F₂C₆H₃ | CO | 1 | S | 92-93 |
| 22 | 2,4-F₂C₆H₃ | CO | 1 | R | |
| 23 | 2,5-F₂C₆H₃ | CO | 1 | S | 128-129 |
| 24 | 2,5-F₂C₆H₃ | CO | 1 | R | |
| 25 | 2-CH₃O—C₆H₄ | CO | 1 | S | |
| 26 | 2,4-(CH₃O)₂C₆H₃ | CO | 1 | S | |
| 27 | 2-O₂N—C₆H₄ | CO | 1 | S | |
| 28 | 3-O₂N—C₆H₄ | CO | 1 | S | |
| 29 | 2-furanyl | CO | 1 | S | |
| 30 | 3-furanyl | CO | 1 | S | |
| 31 | 2-thienyl | CO | 1 | S | |
| 32 | 2-thienyl | CO | 1 | R | |
| 33 | 3-F-thien-2-yl | CO | 1 | S | |
| 34 | 2-F-thien-3-yl | CO | 1 | S | |

¹configuration at the α-carbon
²Here and in the tables below, the numbers mentioned first indicate the binding site of the substituent at the aromatic ring. What follows are the substituent(s) and then the aromatic ring, where C₆Hₙ is a phenyl ring substituted by 5-n groups
³1-C₁₀H₇ = 1-naphthyl, 2-thienyl = thi(oph)en-2-yl, etc.

II Intermediates of the Formula (II) where Y=OR¹, (Hereinbelow Intermediates IIa)

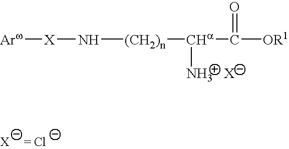

### Intermediate IIa-1: n-Propyl-((S)-N³-(2-fluorobenzoyl)-2,3-diamino)propionate Hydrochloride

At 25° C., 16.3 g of chlorotrimethylsilane (0.15 mol) were added with stirring to a solution of 9.8 g of (S)-N²-(tert-butoxycarbonyl)-N³-(2-fluorobenzoyl)-2,3-diaminopropionic acid (0.03 mol) in 170 ml of n-propanol, and the mixture was stirred at 25° C. for 24 hours. 100 ml of diethyl ether were added, and the precipitated crystals were then filtered off with suction, washed with diethyl ether and pentane and dried. This gave 8 g (87.6% of theory) of n-propyl((S)-N³-(2-fluorobenzoyl)-2,3-diamino)propionate hydrochloride as white crystals of m.p. 187-189° C.

The intermediates IIa-2 to IIa-19 listed in Table 2 were prepared similarly to the preparation of intermediate IIa-1.

TABLE 2
Intermediates of the formula IIa-2 to IIa-19, as hydrochlorides

| Inter- | | | | | | |
|---|---|---|---|---|---|---|
| mediate | | | | | Config. | m.p. |
| IIa-No. | Ar^{ω} | R¹ | X | n | C-α | ° C. |
| 2 | 2-FC₆H₄ | CH₃ | CO | 1 | S | 180- |
| | | | | | | 182 |
| 3 | 2-FC₆H₄ | CH₃ | CO | 1 | R | |
| 4 | 2-FC₆H₄ | C₂H₅ | CO | 1 | S | 186- |
| | | | | | | 188 |
| 5 | 2-FC₆H₄ | C₂H₅ | CO | 1 | R | |
| 6 | 2-FC₆H₄ | C₂H₅ | CO | 1 | rac. | |
| 7 | 2-FC₆H₄ | C₂H₅ | CO | 2 | S | |
| 8 | 2-FC₆H₄ | C₂H₅ | CO | 2 | R | |
| 9 | 2-FC₆H₄ | i-C₃H₇ | CO | 1 | S | 210 |
| 10 | 2-FC₆H₄ | i-C₄H₉ | CO | 1 | S | 186- |
| | | | | | | 188 |
| 1 | 2-FC₆H₄ | i-C₄H₉ | CO | 1 | R | |
| 12 | 2-FC₆H₄ | i-C₄H₉ | CO | 2 | S | |
| 13 | 2-FC₆H₄ | i-C₄H₉ | CO | 4 | S | |
| 14 | 2-FC₆H₄ | i-C₄H₉ | CO | 1 | S | 156- |
| | | | | | | 158 |
| 15 | 2-FC₆H₄ | —(CH₂)₂CH(CH₃)₂ | CO | 1 | S | |
| 16 | 2-FC₆H₄ | —CH₂CH(CH₃)C₂H₅ | CO | 1 | S | |
| 17 | 2-FC₆H₄ | —(CH₂)₃CH(CH₃)₂ | CO | 1 | S | |
| 18 | 2-FC₆H₄ | n-C₆H₁₃ | CO | 1 | S | |
| 19 | 2-FC₆H₄ | —(CH₂)₄CH═CH₂ | CO | 1 | S | |

III Intermediates of the Formula VII where Sg=Butoxycarbonyl

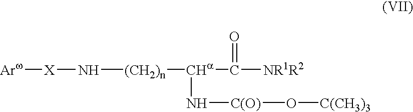

### Intermediate VII-1: N-Methyl-(S)-N³-benzoyl-N²-(tert-butoxycarbonyl)-2,3-diaminopropionamide

16.8 g of 1-ethoxycarbonyl-2-ethoxy-1,2-dihydroquinoline (0.068 mol) and 2.5 g of methylamine (0.08 mol), from a pressurized bottle, were added successively to a solution of 15.4 g of (S)-N³-benzoyl-N²-(tert-butoxycarbonyl)-2,3-diaminopropionic acid (0.05 mol) in 200 ml of tetrahydrofuran. The mixture was stirred at 67° C. for 12 hours and then cooled to −5° C. The resulting precipitate was filtered off with suction, washed successively with tetrahydrofuran, diethyl ether and n-pentane and dried. This gave 15.1 g (93.9% of theory) of N-methyl-(S)-N³-benzoyl-N²-(tert-butoxycarbonyl)-2,3-diaminopropionamide as white crystals of m.p. 222-223° C.

The intermediates VII-2 to VII-54 listed in Table 3 were prepared similarly to the preparation of intermediate VII-1 (Sg=tert-butoxycarbonyl).

TABLE 3
Intermediates of the formula VII-2 to VII-54 as
BOC-protected compounds

| Interme- | | | | | | | |
|---|---|---|---|---|---|---|---|
| diate | | | | | | Config. | |
| VII-No. | Ar^{ω} | R³ | R² | X | n | C-α | m.p. ° C. |
| 2 | 2-CH₃—C₆H₄ | H | CH₃ | CO | 1 | S | 190-191 |
| 3 | 2-CH₃—C₆H₄ | H | CH₃ | CO | 1 | R | |
| 4 | 1-C₁₀H₇ | H | CH₃ | CO | 1 | S | 222-223 |
| 5 | 2-F—C₆H₄ | H | CH₃ | CO | 1 | S | 208-209 |
| 6 | 2-F—C₆H₄ | H | CH₃ | CO | 1 | R | |
| 7 | 2-F—C₆H₄ | H | CH₃ | CO | 1 | rac. | |
| 8 | 2-F—C₆H₄ | H | CH₃ | CO | 2 | S | 143-144 |
| 9 | 2-F—C₆H₄ | H | CH₃ | CO | 2 | R | |
| 10 | 2-F—C₆H₄ | H | CH₃ | CO | 2 | rac. | |
| 11 | 2-F—C₆H₄ | CH₃ | CH₃ | CO | 1 | S | 135-137 |
| 12 | 2-F—C₆H₄ | CH₃ | CH₃ | CO | 1 | R | |
| 13 | 2-F—C₆H₄ | CH₃ | CH₃ | CO | 1 | rac. | |
| 14 | 2-F—C₆H₄ | H | C₂H₅ | CO | 1 | S | 188-189 |
| 15 | 2-F—C₆H₄ | C₂H₅ | C₂H₅ | CO | 1 | S | 113-114 |
| 16 | 2-F—C₆H₄ | H | n-C₃H₇ | CO | 1 | S | 152-153 |
| 17 | 2-F—C₆H₄ | H | i-C₃H₇ | CO | 1 | S | 163-164 |
| 18 | 2-F—C₆H₄ | H | n-C₄H₉ | CO | 1 | S | 172-173 |
| 19 | 2-F—C₆H₄ | H | i-C₄H₉ | CO | 1 | S | 168-169 |
| 20 | 2-F—C₆H₄ | H | tert-C₄H₉ | CO | 1 | S | 128-129 |
| 21 | 2-F—C₆H₄ | H | 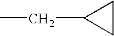 | CO | 1 | S | 179-180 |
| 22 | 2-F—C₆H₄ | H | —CH₂—CH═CH₂ | CO | 1 | S | 148-149 |
| 23 | 2-F—C₆H₄ | H | n-C₅H₁₁ | CO | 1 | S | 152-153 |
| 24 | 2-F—C₆H₄ | H | —(CH₂)₂CH(CH₃)₂ | CO | 1 | S | 146-147 |
| 25 | 2-F—C₆H₄ | H | 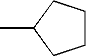 | CO | 1 | S | 189-190 |
| 26 | 2-F—C₆H₄ | H | n-C₆H₁₃ | CO | 1 | S | 138-139 |
| 27 | 2-F—C₆H₄ | H | 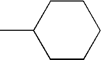 | CO | 1 | S | 187-188 |
| 28 | 2-F—C₆H₄ | H | 2-ETHYL-HEXYL | CO | 1 | S | 149-150 |
| 29 | 2-F—C₆H₄ | H | —(CH₂)₃OC₂H₅ | CO | 1 | S | 122-123 |
| 30 | 2-F—C₆H₄ | —CH₂—CH₂CH₂CH₂ | | CO | 1 | S | 120-121 |
| 31 | 2-F—C₆H₄ | —CH₂—CH₂CH₂CH₂CH₂ | | CO | 1 | S | 119-120 |
| 32 | 2-F—C₆H₄ | H | 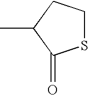 | CO | 1 | S | 184 |
| 33 | 2-F—C₆H₄ | —CH₂—CH₂—O—CH₂CH₂— | | CO | 1 | S | 94-96 |
| 34 | 2-F—C₆H₄ | H | 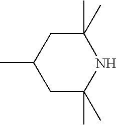 | CO | 1 | S | 146-147 |
| 35 | 2-F—C₆H₄ | H | —C₆H₅ | CO | 1 | S | 205-206 |
| 36 | 2-F—C₆H₄ | H | 2-Cl—C₆H₄ | CO | 1 | S | 189-190 |
| 37 | 2-F—C₆H₄ | H | 4-Cl—C₆H₄ | CO | 1 | S | 194-195 |
| 38 | 2-F—C₆H₄ | H | 2-F—C₆H₄ | CO | 1 | S | |
| 39 | 2-F—C₆H₄ | H | 4-F—C₆H₄ | CO | 1 | S | 174-175 |
| 40 | 2-Cl—C₆H₄ | H | CH₃ | CO | 1 | S | 199-200 |
| 41 | 2-chloropyridin-3-yl | H | CH₃ | CO | 1 | S | 197-198 |
| 42 | 2-Br—C₆H₄ | H | CH₃ | CO | 1 | S | 207-208 |
| 43 | 2,6-F₂C₆H₃ | H | CH₃ | CO | 1 | S | 213-216 |
| 44 | 2,3-F₂C₆H₃ | H | CH₃ | CO | 1 | S | 184-185 |
| 45 | 2,4-F₂C₆H₃ | H | CH₃ | CO | 1 | S | 183-185 |
| 46 | 2,5-F₂C₆H₃ | H | CH₃ | CO | 1 | S | 192-193 |
| 47 | 2-CH₃O—C₆H₄ | H | CH₃ | CO | 1 | S | |
| 48 | 2,4-(CH₃O)₂C₆H₃ | H | CH₃ | CO | 1 | S | |
| 49 | 2-O₂N—C₆H₄ | H | CH₃ | CO | 1 | S | |
| 50 | 3-O₂N—C₆H₄ | H | CH₃ | CO | 1 | S | |
| 51 | 2-FURANYL | H | CH₃ | CO | 1 | S | |
| 52 | 2-THIENYL | H | CH₃ | CO | 1 | S | |
| 53 | 3-F-THIEN-2-YL | H | CH₃ | CO | 1 | S | |
| 54 | 2-F-THIEN-3-YL | H | CH₃ | CO | 1 | S | |

IV Intermediates of the Formula (II) where Y=NR²R³ (Hereinbelow Intermediates IIb) as Hydrochlorides

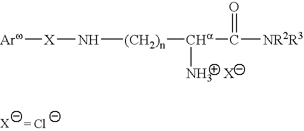

### Intermediate IIb-1: (S)-N-Methyl-(N³-benzoyl-2,3-diamino)propionamide Hydrochloride

At 20-25° C., 150 ml of a 4M solution of hydrogen chloride in dioxane (0.06 mol) were added dropwise to a solution of 13.7 g of N-methyl-(S)-N³-benzoyl-N²-(tert-butoxy-carbonyl)-2,3-diaminopropionamide (0.0426 mol) in 150 ml of dioxane. The mixture was stirred at 20° C. for 14 hours, and the resulting precipitate was then filtered off with suction, washed with diethyl ether and n-pentane and dried. This gave 10.7 g (97.7% of theory) of N-methyl-(N³-benzoyl-2,3-diamino)propionamide hydrochloride as white crystals of m.p. 192-193° C.

The intermediates IIb-2 to IIb-54 listed in Table 4 were prepared similarly to the preparation of intermediate IIb-1.

TABLE 4
Intermediates of the formula IIb-2 to IIb-54 as
hydrochiorides

| Interme- | | | | | | Config. | |
|---|---|---|---|---|---|---|---|
| diate | Ar^{ω} | R² | R³ | X | n | C-α | m.p. ° C. |
| 1 | 2-CH₃C₆H₄ | H | CH₃ | CO | 1 | S | 202-203 |
| 2 | 2-CH₃C₆H₄ | H | CH₃ | CO | 1 | R | |
| 3 | 1-C₁₀H₇ | H | CH₃ | CO | 1 | S | 246-247 |
| 4 | 2-F—C₆H₄ | H | CH₃ | CO | 1 | S | 165-168 |
| 5 | 2-F—C₆H₄ | H | CH₃ | CO | 1 | R | |
| 6 | 2-F—C₆H₄ | H | CH₃ | CO | 1 | rac. | |
| 7 | 2-F—C₆H₄ | H | CH₃ | CO | 2 | S | 210-211 |
| 8 | 2-F—C₆H₄ | H | CH₃ | CO | 2 | R | |
| 9 | 2-F—C₆H₄ | H | CH₃ | CO | 2 | rac. | |
| 10 | 2-F—C₆H₄ | CH₃ | CH₃ | CO | 1 | S | 101-102 |
| 11 | 2-F—C₆H₄ | H | C₂H₅ | CO | 1 | S | 176-177 |
| 12 | 2-F—C₆H₄ | C₂H₅ | C₂H₅ | CO | 1 | S | 91-93 |
| 13 | 2-F—C₆H₄ | H | n-C₃H₇ | CO | 1 | S | 188-189 |
| 14 | 2-F—C₆H₄ | H | i-C₃H₇ | CO | 1 | S | 182-183 |
| 15 | 2-F—C₆H₄ | H | n-C₄H₉ | CO | 1 | S | 192-194 |
| 16 | 2-F—C₆H₄ | H | i-C₄H₉ | CO | 1 | S | 217-218 |
| 17 | 2-F—C₆H₄ | H | tert-C₄H₉ | CO | 1 | S | 123-125 |
| 18 | 2-F—C₆H₄ | H | 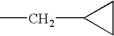 | CO | 1 | S | 201-202 |
| 19 | 2-F—C₆H₄ | H | —CH₂CH═CH₂ | CO | 1 | S | 184-185 |
| 20 | 2-F—C₆H₄ | H | n-C₅H₁₁ | CO | 1 | S | 170-171 |
| 21 | 2-F—C₆H₄ | H | —(CH₂)₂CH(CH₃)₂ | CO | 1 | S | 191-192 |
| 22 | 2-F—C₆H₄ | H | 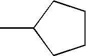 | CO | 1 | S | 178-179 |
| 23 | 2-F—C₆H₄ | H | n-C₆H₁₃ | CO | 1 | S | 172-173 |
| 24 | 2-F—C₆H₄ | H | 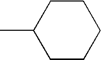 | CO | 1 | S | 178-180 |
| 25 | 2-F—C₆H₄ | H | 2-ethylhex-1-yl | CO | 1 | S | 183-184 |
| 26 | 2-F—C₆H₄ | H | —(CH₂)₃—O—C₂H₅ | CO | 1 | S | 130-131 |
| 27 | 2-F—C₆H₄ | —CH₂—CH₂—CH₂—CH₂— | | CO | 1 | S | 128-129 |
| 28 | 2-F—C₆H₄ | —(CH₂)₅— | | CO | 1 | S | 132-133 |
| 29 | 2-F—C₆H₄ | H | 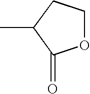 | CO | 1 | S | 211-212 |
| 30 | 2-F—C₆H₄ | H | 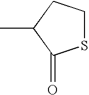 | CO | 1 | S | 188-189 |
| 31 | 2-F—C₆H₄ | —CH₂—CH₂—O—CH₂—CH₂— | | CO | 1 | S | 158-159 |
| 32 | 2-F—C₆H₄ | H | C₆H₅ | CO | 1 | S | 239-240 |
| 33 | 2-F—C₆H₄ | H | 2-Cl—C₆H₄ | CO | 1 | S | 239-241 |
| 34 | 2-F—C₆H₄ | H | 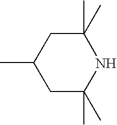 | CO | 1 | S | 242-243 |
| 35 | 2-F—C₆H₄ | H | 4-Cl—C₆H₄ | CO | 1 | S | 257-258 |
| 36 | 2-F—C₆H₄ | H | 4-F—C₆H₄ | CO | 1 | S | 254-255 |
| 37 | 2-F—C₆H₄ | H | 2-F—C₆H₄ | CO | 1 | S | |
| 38 | 2-Cl—C₆H₄ | H | CH₃ | CO | 1 | S | 191-192 |
| 39 | 2-Br—C₆H₄ | H | CH₃ | CO | 1 | S | 212-213 |
| 40 | 2-chloropyridin-3-yl | H | CH₃ | CO | 1 | S | 205-206 |
| 41 | 2,6-F₂—C₆H₃ | H | CH₃ | CO | 1 | S | 225-228 |
| 42 | 2,3-F₂—C₆H₃ | H | CH₃ | CO | 1 | S | 110-112 |
| 43 | 2,4-F₂—C₆H₃ | H | CH₃ | CO | 1 | S | 169-170 |
| 44 | 2,5-F₂—C₆H₃ | H | CH₃ | CO | 1 | S | 178-179 |
| 45 | 2-CH₃O—C₆H₄ | H | CH₃ | CO | 1 | S | |
| 46 | 2,4(CH₃O)₂C₆H₃ | H | CH₃ | CO | 1 | S | |
| 47 | 2-O₂N—C₆H₄ | H | CH₃ | CO | 1 | S | |
| 48 | 3-O₂N—C₆H₄ | H | CH₃ | CO | 1 | S | |
| 49 | 2-FURANYL | H | CH₃ | CO | 1 | S | |
| 50 | 2-THIENYL | H | CH₃ | CO | 1 | S | |
| 51 | 3-F-thiophen- | H | CH₃ | CO | 1 | S | |
| | 2-yl | | | | | | |
| 52 | 2-F-thiophen- | H | CH₃ | CO | 1 | S | |
| | 3-yl | | | | | | |
| 53 | 2-F—C₆H₄ | H | CH₃ | CO | 4 | S | |
| 54 | 2-F—C₆H₄ | H | CH₃ | CO | 4 | S | |

V Compounds of the Formula I where Y=OR¹ (Hereinbelow Compounds Ia)

### Example 1

### Ethyl (S)-(N³-(2-fluorobenzoyl)-N²-(2-chlorobenzoyl)-2,3-diamino)propionate (Compound Ia-1)

1.4 g of N-ethyldiisopropylamine (0.011 mol) and then, at from −10° C. to −5° C., a solution of 0.9 g of 2-chlorobenzoyl chloride (0.005 mol) in 10 ml of methylene chloride were successively added dropwise to a solution of 1.5 g of ethyl (S)-(N³-(2-fluorobenzoyl)-2,3-diamino)propionate (0.005 mol) in 20 ml of methylene chloride. The mixture was stirred at −10° C. for 2 hours and then at 22° C. for another 14 hours. The resulting mixture was subsequently washed twice with in each case 50 ml of water. The organic phase was separated off and concentrated under reduced pressure and the residue was stirred with diethyl ether. The resulting precipitate was filtered off with suction and washed with a little diethyl ether and n-pentane. This gave 1.4 g (71.4% of theory) of ethyl (S)-(N³-(2-fluorobenzoyl)-N²-(2-chlorobenzoyl)-2,3-diamino)propionate as white crystals of m.p. 107-110° C.

The active compounds of the formula Ia listed in Table 5 were prepared similarly to the preparation of the compound Ia-1 (Examples 1 to 40).

VI Compounds of the Formula I where Y=NR²R³ (Hereinbelow Compounds Ib)

### Example 41

### (S)-N-Methyl-((N³-benzoyl)-N²-(2,4-dichlorobenzoyl)-2,3-diamino)propionamide

2.6 g (0.025 mol) of triethylamine and then, at from −5 to −10° C., a solution of 2.1 g (0.01 mol) of 2,4-dichlorobenzoyl chloride in 30 ml of methylene chloride were successively added dropwise to a solution of 2.6 g of (S)-N-methyl-((N³-benzoyl)-2,3-diamino)propionamide (0.01 mol) in 40 ml of methylene chloride. The mixture was stirred at −5° C. for 2 hours and then at 22° C. for another 14 hours. The resulting precipitate was filtered off with suction, washed successively with methylene chloride, water, methanol and diethyl ether and dried under reduced pressure. This gave 2.5 g (63.4% of theory) of (S)-N-methyl-((N³-benzoyl-N²-(2,4-dichlorobenzoyl)-2,3-diamino)propionamide as white crystals of m.p. 224-225° C.

The active compounds of the formula Ib listed in Table 6 were prepared similarly to Example 41 (Examples 41 to 200).

TABLE 5
Compounds of the formula Ia (Examples 1 to 40)

| | |
|---|---|
| | (I) |
| 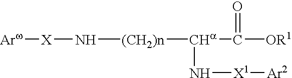 | |

| Exam- | | | | | | | | |
|---|---|---|---|---|---|---|---|---|
| ple | Ar^{ω} | Ar² | R¹ | X | X¹ | n | Config. at C = α | m.p. ° C. |
| 1 | 2-F—C₆H₄ | 2-Cl—C₆H₄ | CH₂CH₃ | CO | CO | 1 | S | 107-110 |
| 2 | 2-F—C₆H₄ | 2-Cl—C₆H₄ | CH₃ | CO | CO | 1 | S | 155-157 |
| 3 | 2-F—C₆H₄ | 2-Cl—C₆H₄ | CH₃ | CO | CO | 1 | R | |
| 4 | 2-F—C₆H₄ | 2-Cl—C₆H₄ | CH₃ | CO | SO₂ | 1 | S | |
| 5 | 2-F—C₆H₄ | 2-Br—C₆H₄ | CH₃ | CO | CO | 1 | S | 150-153 |
| 6 | 2-F—C₆H₄ | 2,4-Cl₂C₆H₃ | CH₃ | CO | CO | 1 | S | 168-170 |
| 7 | 2-F—C₆H₄ | 2,3-Cl₂C₆H₃ | CH₃ | CO | CO | 1 | S | 154-157 |
| 8 | 2-F—C₆H₄ | 2,3-Cl₂C₆H₃ | CH₃ | CO | CO | 1 | R | |
| 9 | 2-F—C₆H₄ | 2,3-Cl₂C₆H₃ | CH₃ | CO | CO | 1 | S | |
| 10 | 2-F—C₆H₄ | 2,3-Cl₂C₆H₃ | CH₃ | CO | CO | 1 | S | |
| 11 | 2-F—C₆H₄ | 2,3-Cl₂C₆H₃ | CH₃ | CO | CO | 1 | S | |
| 12 | 2-F—C₆H₄ | 2-BrC₆H₄ | C₂H₅ | CO | CO | 1 | S | 118-120 |
| 13 | 2-F—C₆H₄ | 2,4-Cl₂C₆H₃ | C₂H₅ | CO | CO | 1 | S | 147-148 |
| 14 | 2-F—C₆H₄ | 2,3-Cl₂C₆H₃ | C₂H₅ | CO | CO | 1 | S | 150-151 |
| 15 | 2-F—C₆H₄ | 2,3,4-Cl₃C₆H₂ | C₂H₅ | CO | CO | 1 | S | |
| 16 | 2-F—C₆H₄ | 2-ClC₆H₄ | n-C₃H₇ | CO | CO | 1 | S | 103-104 |
| 17 | 2-F—C₆H₄ | 2-BrC₆H₄ | n-C₃H₇ | CO | CO | 1 | S | 121-122 |
| 18 | 2-F—C₆H₄ | 2,4-Cl₂C₆H₃ | n-C₃H₇ | CO | CO | 1 | S | 116-118 |
| 19 | 2-F—C₆H₄ | 2,3-Cl₂C₆H₃ | n-C₃H₇ | CO | CO | 1 | S | 142-143 |
| 20 | 2-F—C₆H₄ | 2,3-Cl₂C₆H₃ | n-C₃H₇ | CO | CO | 1 | S | |
| 21 | 2-F—C₆H₄ | 2,3,4-Cl₂C₆H₂ | n-C₃H₇ | CO | CO | 1 | S | |
| 22 | 2-F—C₆H₄ | 2,3,4-Cl₃C₆H₂ | n-C₃H₇ | CO | CO | 1 | R | |
| 23 | 2-F—C₆H₄ | 2-ClC₆H₄ | i-C₃H₇ | CO | CO | 1 | S | 122-125 |
| 24 | 2-F—C₆H₄ | 2,4-Cl₂C₆H₃ | i-C₃H₇ | CO | CO | 1 | S | 83 |
| 25 | 2-F—C₆H₄ | 2,3-Cl₂C₆H₃ | i-C₃H₇ | CO | CO | 1 | S | 113-116 |
| 26 | 2-F—C₆H₄ | 2,3,4-Cl₃C₆H₂ | i-C₃H₇ | CO | CO | 1 | S | |
| 27 | 2-F—C₆H₄ | 2-ClC₆H₄ | n-C₄H₉ | CO | CO | 1 | S | 105-108 |
| 28 | 2-F—C₆H₄ | 2,4-Cl₂C₆H₃ | n-C₄H₉ | CO | CO | 1 | S | 123-125 |
| 29 | 2-F—C₆H₄ | 2,3-Cl₂C₆H₃ | n-C₄H₉ | CO | CO | 1 | S | 130-132 |
| 30 | 2-F—C₆H₄ | 2,3,4-Cl₃C₆H₂ | n-C₄H₉ | CO | CO | 1 | S | |
| 31 | 2-F—C₆H₄ | 2-Cl—C₆H₄ | i-C₄H₉ | CO | CO | 1 | S | 102 |
| 32 | 2-F—C₆H₄ | 2-BrC₆H₄ | i-C₄H₉ | CO | CO | 1 | S | 103-106 |
| 33 | 2-F—C₆H₄ | 2,4-Cl₂C₆H₃ | i-C₄H₉ | CO | CO | 1 | S | 113-115 |
| 34 | 2-F—C₆H₄ | 2,3-Cl₂C₆H₃ | i-C₄H₉ | CO | CO | 1 | S | 136-138 |
| 35 | 2-F—C₆H₄ | 2,3-Cl₂C₆H₃ | n-C₅H₁₁ | CO | CO | 1 | S | |
| 36 | 2-F—C₆H₄ | 2,3-Cl₂C₆H₃ | —(CH₂)₂CH(CH₃)₃ | CO | CO | 1 | S | |
| 37 | 2-F—C₆H₄ | 2,3-Cl₂C₆H₃ | -n-C₆H₁₃ | CO | CO | 1 | S | |
| 38 | 2-F—C₆H₄ | 2,3-Cl₂C₆H₃ | —(CH₂)₃CH(CH₃)₂ | CO | CO | 1 | S | |
| 39 | 2-F—C₆H₄ | 2,3-Cl₂C₆H₃ | —(CH₂)₃CH═CH₂ | CO | CO | 1 | S | |
| 40 | 2-F—C₆H₄ | 2,3-Cl₂C₆H₃ | —CH₂CH(CH₃)C₂H₅ | CO | CO | 1 | S | |

TABLE 6
Compounds of the formula Ib (Examples 41 to 200)

| | |
|---|---|
| | (Ib) |
| 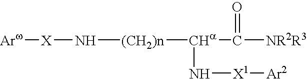 | |

| Ex. | Ar^{ω} | Ar² | R³ | R² | X | X¹ | n | Config. at C = α | m.p. ° C. |
|---|---|---|---|---|---|---|---|---|---|
| 41 | C₆H₅ | 2,4-Cl₂C₆H₃ | H | CH₃ | CO | CO | 1 | S | 224-225 |
| 42 | 2-CH₃—C₆H₄ | 2-ClC₆H₄ | H | CH₃ | CO | CO | 1 | S | 257-258 |
| 43 | 2-CH₃—C₆H₄ | 2,4-Cl₂C₆H₃ | H | CH₃ | CO | CO | 1 | S | 254-255 |
| 44 | 1-C₁₀H₇ | 2-ClC₆H₄ | H | CH₃ | CO | CO | 1 | S | 256-257 |
| 45 | 2-ClC₆H₄ | 2-ClC₆H₄ | H | CH₃ | CO | CO | 1 | S | 237-238 |
| 46 | 2-ClC₆H₄ | 2,4F₂C₆H₃ | H | CH₃ | CO | CO | 1 | S | 238-239 |
| 47 | 2-ClC₆H₄ | 2,4Cl₂C₆H₃ | H | CH₃ | CO | CO | 1 | S | 250-251 |
| 48 | 2-BrC₆H₄ | 2-FC₆H₄ | H | CH₃ | CO | CO | 1 | S | 234-235 |
| 49 | 2-BrC₆H₄ | 2-ClC₆H₄ | H | CH₃ | CO | CO | 1 | S | 246-247 |
| 50 | 2-BrC₆H₄ | 2,4-Cl₂C₆H₃ | H | CH₃ | CO | CO | 1 | S | 261-262 |
| 51 | 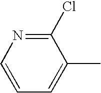 | 2-ClC₆H₄ | H | CH₃ | CO | CO | 1 | S | 268-269 |
| 52 | ″ | 2,4-Cl₂C₆H₃ | H | CH₃ | CO | CO | 1 | S | 256-257 |
| 53 | 2-FC₆H₄ | 2-CH₃C₆H₄ | H | CH₃ | CO | CO | 1 | S | 213-216 |
| 54 | 2-FC₆H₄ | 2,3-(CH₃)₂C₆H₃ | H | CH₃ | CO | CO | 1 | S | 235-237 |
| 55 | 2-FC₆H₄ | 2,4,6-(CH₃)₃C₆H₂ | H | CH₃ | CO | SO₂ | 1 | S | 190-193 |
| 56 | 2-FC₆H₄ | 2,3,6(CH₃)₃-4-(CH₃O)C₆H₄ | H | CH₃ | CO | SO₂ | 1 | S | 193-196 |
| 57 | 2-FC₆H₄ | 4-(C₄H₉)C₆H₄ | H | CH₃ | CO | CO | 1 | 5 | 225-228 |
| 58 | 2-FC₆H₄ | 4-(C₆H₁₃)C₆H₄ | H | CH₃ | CO | CO | 1 | S | 195-198 |
| 59 | 2-FC₆H₄ | 4-(C₇H₁₅)C₆H₄ | H | CH₃ | CO | CO | 1 | S | 190-194 |
| 60 | 2-FC₆H₄ | 1-C₁₀H₇ | H | CH₃ | CO | CO | 1 | S | 235-236 |
| 61 | 2-FC₆H₄ | 1-C₁₀H₇ | H | CH₃ | CO | SO₂ | 1 | S | 212-214 |
| 62 | 2-FC₆H₄ | 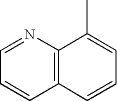 | H | CH₃ | CO | CO | 1 | S | 129-132 |
| 63 | 2-FC₆H₄ | 2(CH₃COO)C₆H₄ | H | CH₃ | CO | CO | 1 | S | 211-212 |
| 64 | 2-FC₆H₄ | 2(CH₃O)C₆H₄ | H | CH₃ | CO | CO | 1 | S | 170-172 |
| 65 | 2-FC₆H₄ | 2,4(CH₃O)₂C₆H₃ | H | CH₃ | CO | CO | 1 | S | 203-205 |
| 66 | 2-FC₆H₄ | 2-FC₆H₄ | H | CH₃ | CO | CO | 1 | S | 215-217 |
| 67 | 2-FC₆H₄ | 2,4-F₂C₆H₃ | H | CH₃ | CO | CO | 1 | S | 205-207 |
| 68 | 2-FC₆H₄ | 2,3-F₂C₆H₃ | H | CH₃ | CO | CO | 1 | S | 225-227 |
| 69 | 2-FC₆H₄ | 2,4,5-F₃C₆H₂ | H | CH₃ | CO | CO | 1 | S | 207-209 |
| 70 | 2-FC₆H₄ | 2-F,4-ClC₆H₃ | H | CH₃ | CO | CO | 1 | S | 232-233 |
| 71 | 2-FC₆H₄ | 2-(CF₃)C₆H₄ | H | CH₃ | CO | CO | 1 | S | 249-251 |
| 72 | 2-FC₆H₄ | 2-ClC₆H₄ | H | CH₃ | CO | CO | 1 | S | 210-212 |
| 73 | 2-FC₆H₄ | 3-ClC₆H₄ | H | CH₃ | CO | CO | 1 | S | 222-224 |
| 74 | 2-FC₆H₄ | 4-ClC₆H₄ | H | CH₃ | CO | CO | 1 | S | 245-248 |
| 75 | 2-FC₆H₄ | 2,6-Cl₂C₆H₃ | H | CH₃ | CO | CO | 1 | S | 256-259 |
| 76 | 2-FC₆H₄ | 2,4-Cl₂C₆H₃ | H | CH₃ | CO | CO | 1 | S | 239-241 |
| 77 | 2-FC₆H₄ | 2,4-Cl₂C₆H₃ | H | CH₃ | CO | CO | 1 | R | |
| 78 | 2-FC₆H₄ | 2,4-Cl₂C₆H₃ | H | CH₃ | CO | CO | 1 | rac. | |
| 79 | 2-FC₆H₄ | 2,4-Cl₂C₆H₃ | H | CH₃ | CO | CO | 2 | S | 224-225 |
| 80 | 2-FC₆H₄ | 2,4-Cl₂C₆H₃ | H | CH₃ | CO | CO | 2 | R | |
| 81 | 2-FC₆H₄ | 2,4-Cl₂C₆H₃ | H | CH₃ | CO | CO | 3 | S | |
| 82 | 2-FC₆H₄ | 2,4-Cl₂C₆H₃ | H | CH₃ | CO | CO | 4 | S | |
| 83 | 2-FC₆H₄ | 2,4-Cl₂C₆H₃ | H | CH₃ | CO | CO | 4 | R | |
| 84 | 2-FC₆H₄ | 2,4-Cl₂C₆H₃ | H | C₂H₅ | CO | CO | 4 | S | |
| 85 | 2-FC₆H₄ | 2,5-Cl₂C₆H₃ | H | CH₃ | CO | CO | 1 | S | 236-238 |
| 86 | 2-FC₆H₄ | 2,5-Cl₂06H3 | H | CH₃ | CO | CO | 1 | S | 198-200 |
| 87 | 2-FC₆H₄ | 2-OH,3,5-Cl₂C₆H₂ | H | CH₃ | CO | CO | 1 | S | 156 |
| 88 | 2-FC₆H₄ | 2,3-Cl₂C₆H₃ | H | CH₃ | CO | CO | 1 | S | 241-244 |
| 89 | 2-FC₆H₄ | 2,3-Cl₂C₆H₃ | H | CH₃ | CO | CO | 1 | R | |
| 90 | 2-FC₆H₄ | 2,3-Cl₂C₆H₃ | H | CH₃ | CO | CO | 1 | rac. | |
| 91 | 2-FC₆H₄ | 2,3-Cl₂C₆H₃ | H | CH₃ | CO | CO | 2 | S | 229-230 |
| 92 | 2-FC₆H₄ | 2,3-Cl₂C₆H₃ | H | CH₃ | CO | CO | 2 | rac. | |
| 93 | 2-FC₆H₄ | 2,3-Cl₂C₆H₃ | H | CH₃ | CO | CO | 4 | S | |
| 94 | 2-FC₆H₄ | 2,3-Cl₂C₆H₃ | H | C₂H₅ | CO | CO | 4 | S | |
| 95 | 2-FC₆H₄ | 2,4-Cl₂C₆H₃ | CH₃ | CH₃ | CO | CO | 1 | S | 150-151 |
| 96 | 2-FC₆H₄ | 2,4-Cl₂C₆H₃ | H | C₂H₅ | CO | CO | 1 | S | 221-222 |
| 97 | 2-FC₆H₄ | 2,3-Cl₂C₆H₃ | H | C₂H₅ | CO | CO | 1 | S | 255-256 |
| 98 | 2-FC₆H₄ | 2,4-Cl₂C₆H₃ | C₂H₅ | C₂H₅ | CO | CO | 1 | S | 59-61 |
| 99 | 2-FC₆H₄ | 2,4-Cl₂C₆H₃ | H | n-C₃H₇ | CO | CO | 1 | S | 204-205 |
| 100 | 2-FC₆H₄ | 2,3-Cl₂C₆H₃ | H | n-C₃H₇ | CO | CO | 1 | S | 219-220 |
| 101 | 2-FC₆H₄ | 2,4-Cl₂C₆H₃ | H | —CH₂—CH═CH₂ | CO | CO | 1 | S | 200-201 |
| 102 | 2-FC₆H₄ | 2,3-Cl₂C₆H₃ | H | —CH₂—CH═CH₂ | CO | CO | 1 | S | 218-219 |
| 103 | 2-FC₆H₄ | 2,4-Cl₂C₆H₃ | H | i-C₃H₇ | CO | CO | 1 | S | 224-225 |
| 104 | 2-FC₆H₄ | 2,3-Cl₂C₆H₃ | H | i-C₃H₇ | CO | CO | 1 | S | 238-239 |
| 105 | 2-FC₆H₄ | 2,4-Cl₂C₆H₃ | H | n-C₄H₉ | CO | CO | 1 | S | 213-214 |
| 106 | 2-FC₆H₄ | 2,3-Cl₂C₆H₃ | H | n-C₄H₉ | CO | CO | 1 | S | 204-205 |
| 107 | 2-FC₆H₄ | 2,4-Cl₂C₆H₃ | H | i-C₄H₉ | CO | CO | 1 | S | 227-228 |
| 108 | 2-FC₆H₄ | 2,3-Cl₂C₆H₃ | H | i-C₄H₉ | CO | CO | 1 | S | 212-213 |
| 109 | 2-FC₆H₄ | 2,4-Cl₂C₆H₃ | H | tert-C₄H₉ | CO | CO | 1 | S | 162-163 |
| 110 | 2-FC₆H₄ | 2,3-Cl₂C₆H₃ | H | tert-C₄H₉ | CO | CO | 1 | S | 200-201 |
| 111 | 2-FC₆H₄ | 2,4-Cl₂C₆H₃ | H | 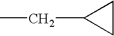 | CO | CO | 1 | S | 218-219 |
| 112 | 2-FC₆H₄ | 2,3-Cl₂C₆H₃ | H | 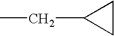 | CO | CO | 1 | S | 211-212 |
| 113 | 2-FC₆H₄ | 2,4-Cl₂C₆H₃ | H | n-C₅H₁₁ | CO | CO | 1 | S | 209-210 |
| 114 | 2-FC₆H₄ | 2,3-Cl₂C₆H₃ | H | n-C₅H₁₁ | CO | CO | 1 | S | 217-218 |
| 115 | 2-FC₆H₄ | 2,4-Cl₂C₆H₃ | H | —(CH₂)₂CH(CH₃)₂ | CO | CO | 1 | S | 207-208 |
| 116 | 2-FC₆H₄ | 2,3-Cl₂C₆H₃ | H | —(CH₂)₂CH(CH₃)₂ | CO | CO | 1 | S | 203-204 |
| 117 | 2-FC₆H₄ | 2,4-Cl₂C₆H₃ | H | CYCLO-C₅H₉ | CO | CO | 1 | S | 239-240 |
| 118 | 2-FC₆H₄ | 2,3-Cl₂C₆H₃ | H | CYCLO-C₅H₉ | CO | CO | 1 | S | 196-197 |
| 119 | 2-FC₆H₄ | 2,4-Cl₂C₆H₃ | H | n-C₆H₁₃ | CO | CO | 1 | S | 203-204 |
| 120 | 2-FC₆H₄ | 2,3-Cl₂C₆H₃ | H | n-C₆H₃ | CO | CO | 1 | S | 209-211 |
| 121 | 2-FC₆H₄ | 2,4-Cl₂C₆H₃ | H | CYCLO-C₆H₁₁ | CO | CO | 1 | S | 250-251 |
| 122 | 2-FC₆H₄ | 2,3-Cl₂C₆H₃ | H | CYCLO-C₆H₁₁ | CO | CO | 1 | S | 209-210 |
| 123 | 2-FC₆H₄ | 2,4-Cl₂C₆H₃ | H | —CH₂CH(C₂H₅)C₄H₉ | CO | CO | 1 | 5 | 172-173 |
| 124 | 2-FC₆H₄ | 2,3-Cl₂C₆H₃ | H | —CH₂CH(C₂H₅)C₄H₉ | CO | CO | 1 | 5 | 161-162 |
| 125 | 2-FC₆H₄ | 2,4-Cl₂C₆H₃ | H | —(CH₂)₃—O—C₂H₅ | CO | CO | 1 | 5 | 178-179 |
| 126 | 2-FC₆H₄ | 2,3-Cl₂C₆H₃ | H | —(CH₂)₃—O—C₂H₅ | CO | CO | 1 | S | 181-182 |
| 127 | 2-FC₆H₄ | 2,4-Cl₂C₆H₃ | —CH₂—CH₂—CH₂—CH₂ | | CO | CO | 1 | S | 141-142 |
| 128 | 2-FC₆H₄ | 2,3-Cl₂C₆H₃ | —CH₂—CH₂—CH₂—CH₂ | | CO | CO | 1 | S | 148-149 |
| 129 | 2-FC₆H₄ | 2,4-Cl₂C₆H₃ | —CH₂—CH₂—CH₂—CH₂—CH₂ | | CO | CO | 1 | S | 126-127 |
| 130 | 2-FC₆H₄ | 2,3-Cl₂C₆H₃ | —CH₂—CH₂—CH₂—CH₂—CH₂ | | CO | CO | 1 | S | 101-102 |
| 131 | 2-FC₆H₄ | 2,4-Cl₂C₆H₃ | —CH₂—CH₂—O—CH₂—CH₂ | | CO | CO | 1 | S | 152-153 |
| 132 | 2-FC₆H₄ | 2,3-Cl₂C₆H₃ | —CH₂—CH₂—O—CH₂—CH₂ | | CO | CO | 1 | S | 156-157 |
| 133 | 2-FC₆H₄ | 2,4-Cl₂C₆H₃ | H | 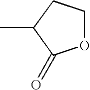 | CO | CO | 1 | S | 224-225 |
| 134 | 2-FC₆H₄ | 2,4-Cl₂C₆H₃ | H | 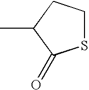 | CO | CO | 1 | S | 219-220 |
| 135 | 2-FC₆H₄ | 2,3-Cl₂C₆H₃ | H | ″ | CO | CO | 1 | S | 209-210 |
| 136 | 2-FC₆H₄ | 24-Cl₂C₆H₃ | H | 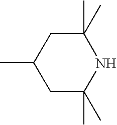 | CO | CO | 1 | S | 253-254 |
| 137 | 2-FC₆H₄ | 2,3-Cl₂C₆H₃ | H | ″ | CO | CO | 1 | S | 142-143 |
| 138 | 2-FC₆H₄ | 2,4-Cl₂C₆H₃ | H | C₆H₅ | CO | CO | 1 | S | 239-240 |
| 139 | 2-FC₆H₄ | 2,3-Cl₂C₆H₃ | H | C₆H₅ | CO | CO | 1 | S | 250-251 |
| 140 | 2-FC₆H₄ | 2,4-Cl₂C₆H₃ | H | 2-ClC₆H₄ | CO | CO | 1 | S | 229-230 |
| 141 | 2-FC₆H₄ | 2,3-Cl₂C₆H₃ | H | 2-ClC₆H₄ | CO | CO | 1 | S | 224-225 |
| 142 | 2-FC₆H₄ | 2,4-Cl₂C₆H₃ | H | 4-ClC₆H₄ | CO | CO | 1 | S | 239-240 |
| 143 | 2-FC₆H₄ | 2,3-Cl₂C₆H₃ | H | 4-ClC₆H₄ | CO | CO | 1 | S | 261-262 |
| 144 | 2-FC₆H₄ | 2,4-Cl₂C₆H₃ | H | 4-FC₆H₄ | CO | CO | 1 | S | 235-236 |
| 145 | 2-FC₆H₄ | 2,3-Cl₂C₆H₃ | H | 4-FC₆H₄ | CO | CO | 1 | S | 245-246 |
| 146 | 2-FC₆H₄ | 2-NH₂,4ClC₆H₃ | H | CH₃ | CO | CO | 1 | S | 206-209 |
| 147 | 2-FC₆H₄ | 2-NH₂,5CH₃C₆H₃ | H | CH₃ | CO | CO | 1 | S | 216-218 |
| 148 | 2-FC₆H₄ | 2-NH₂,6FC₆H₃ | H | CH₃ | CO | CO | 1 | S | 223-225 |
| 149 | 2-FC₆H₄ | 2-NH₂,5ClC₆H₃ | H | CH₃ | CO | CO | 1 | S | 233-236 |
| 150 | 2-FC₆H₄ | 2-NH₂,3,5Cl₂C₆H₂ | H | CH₃ | CO | CO | 1 | S | 257-260 |
| 151 | 2-FC₆H₄ | 2-NH₂C₆H₄ | H | CH₃ | CO | CO | 1 | S | 219-222 |
| 152 | 2-FC₆H₄ | 2-NH₂,4HOC₆H₃ | H | CH₃ | CO | CO | 1 | S | 276-279 |
| 153 | 2-FC₆H₄ | 2-NO₂C₆H₄ | H | CH₃ | CO | CO | 1 | S | 247-249 |
| 154 | 2-FC₆H₄ | 2-NO₂C₆H₄ | H | CH₃ | CO | CO | 1 | S | 174-175 |
| 155 | 2-FC₆H₄ | 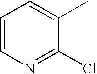 | H | CH₃ | CO | CO | 1 | S | 234-235 |
| 156 | 2-FC₆H₄ | 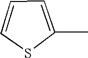 | H | CH₃ | CO | CO | 1 | S | 200-203 |
| 157 | 2-FC₆H₄ | 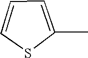 | H | CH₃ | CO | SO₂ | 1 | S | 216-218 |
| 158 | 2-FC₆H₄ | 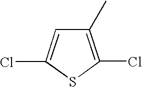 | H | CH₃ | CO | CO | 1 | S | 212-213 |
| 159 | 2-FC₆H₄ | 2-BrC₆H₄ | H | CH₃ | CO | CO | 1 | S | 210-212 |
| 160 | 2-FC₆H₄ | 2-BrC₆H₄ | H | CH₃ | CO | CO | 2 | S | 209-211 |
| 161 | 2-FC₆H₄ | 3-BrC₆H₄ | H | CH₃ | CO | CO | 1 | S | 220-223 |
| 162 | 2-FC₆H₄ | 2-IC₆H₄ | H | CH₃ | CO | CO | 1 | S | 226-229 |
| 163 | 2,6-F₂C₆H₃ | 4(C₄H₉)C₆H₄ | H | CH₃ | CO | CO | 1 | S | 263-265 |
| 164 | 2,6-F₂C₆H₃ | 4(C₆H₁₃)C₆H₄ | H | CH₃ | CO | CO | 1 | S | 239-242 |
| 165 | 2,6-F₂C₆H₃ | 4(C₇H₁₅)C₆H₄ | H | CH₃ | CO | CO | 1 | S | 226-228 |
| 166 | 2,6-F₂C₆H₃ | 2-ClC₆H₄ | H | CH₃ | CO | CO | 1 | S | 231-234 |
| 167 | 2,6-F₂C₆H₃ | 4-ClC₆H₄ | H | CH₃ | CO | CO | 1 | S | 275 |
| 168 | 2,6-F₂C₆H₃ | 2,4-Cl₂C₆H₃ | H | CH₃ | CO | CO | 1 | S | 257-260 |
| 169 | 2,6-F₂C₆H₃ | 2,6-Cl₂C₆H₃ | H | CH₃ | Co | CO | 1 | S | 292-294 |
| 170 | 2,6-F₂C₆H₃ | 2-IC₆H₄ | H | CH₃ | CO | CO | 1 | S | 256-257 |
| 171 | 2,4-F₂C₆H₃ | 2,4-Cl₂C₆H₃ | H | CH₃ | CO | CO | 1 | S | 231-232 |
| 172 | 2,3-F₂C₆H₃ | 2,4-Cl₂C₆H₃ | H | CH₃ | CO | CO | 1 | S | 232-233 |
| 173 | 2,5-F₂C₆H₃ | 2,4-Cl₂C₆H₃ | H | CH₃ | CO | CO | 1 | S | 216-217 |
| 174 | 2-FC₆H₄ | 2,3-Cl₂C₆H₃ | CH₃ | CH₃ | CO | CO | 1 | S | 145-146 |
| 175 | 2-FC₆H₄ | 2,3-Cl₂C₆H₃ | H | 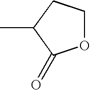 | CO | CO | 1 | S | 256-257 |
| 176 | 2-FC₆H₄ | 2,3-Cl₂C₆H₃ | C₂H₅ | C₂H₅ | CO | CO | 1 | S | 40-42 |
| 177 | 2-FC₆H₄ | 2,4-Cl₂C₆H₃ | H | 2-FC₆H₄ | CO | CO | 1 | S | 226-227 |
| 178 | 2-FC₆H₄ | 2,3-Cl₂C₆H₃ | H | 2-FC₆H₄ | CO | CO | 1 | S | 239-240 |
| 179 | 2-FC₆H₄ | 2-BrC₆H₄ | H | 2-FC₆H₄ | CO | CO | 1 | S | 193-194 |
| 180 | 2-FC₆H₄ | 2,3,4-Cl₃C₆H₂ | H | C₂H₅ | CO | CO | 1 | S | 258-259 |
| 181 | 2-FC₆H₄ | 2,3,4-Cl₃C₆H₂ | H | n-C₃H₇ | CO | CO | 1 | S | 231-232 |
| 182 | 2-FC₆H₄ | 2,3,4-Cl₃C₆H₂ | H | iso-C₃H₇ | CO | CO | 1 | 5 | 254-255 |
| 183 | 2-FC₆H₄ | 2,3,4-Cl₃C₆H₂ | H | n-C₄H₉ | CO | CO | 1 | S | 219-220 |
| 184 | 2-FC₆H₄ | 2,3,4-Cl₃C₆H₂ | H | CH₂CH(CH₃)₂ | CO | CO | 1 | S | 229-230 |
| 185 | 2-FC₆H₄ | 2,3,4-Cl₃C₆H₂ | H | tert-C₄H₉ | CO | CO | 1 | S | 184-185 |
| 186 | 2-FC₆H₄ | 2,3,4-Cl₃C₆H₂ | H | 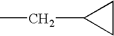 | CO | CO | 1 | 5 | 230-231 |
| 187 | 2-FC₆H₄ | 2,3,4-Cl₃C₆H₂ | H | CH₂CH═CH₂ | CO | CO | 1 | S | 226-227 |
| 188 | 2-FC₆H₄ | 2,3,4-Cl₃C₆H₂ | H | n-C₅H₁₁ | CO | CO | 1 | S | 220-221 |
| 189 | 2-FC₆H₄ | 2,3,4-Cl₃C₆H₂ | H | CH₂CH₂CH(CH₃)₂ | CO | CO | 1 | S | 214-215 |
| 190 | 2-FC₆H₄ | 2,3,4-Cl₃C₆H₂ | H | n-C₆H₁₃ | CO | CO | 1 | S | 212-213 |
| 191 | 2-FC₆H₄ | 2,3,4-Cl₃C₆H₂ | H | 2-ethylhex-1-yl | CO | CO | 1 | S | 107-198 |
| 192 | 2-FC₆H₄ | 2,3,4-Cl₃C₆H₂ | H | CH₂CH₂CH₂OCH₂CH₃ | CO | CO | 1 | S | 199-200 |
| 193 | 2-FC₆H₄ | 2,3,4-Cl₃C₆H₂ | H | cyclohexyl | CO | CO | 1 | S | 238-239 |
| 194 | 2-FC₆H₄ | 2,3,4-Cl₃C₆H₂ | H | cyclopentyl | CO | CO | 1 | S | 237-238 |
| 195 | 2-FC₆H₄ | 2,3,4-Cl₃C₆H₂ | —CH₂—CH₂—CH₂—CH₂ | | CO | CO | 1 | S | 137-138 |
| 196 | 2-FC₆H₄ | 2,3,4-Cl₃C₆H₂ | —CH₂—CH₂—CH₂—CH₂—CH₂ | | CO | CO | 1 | S | 112-114 |
| 197 | 2-FC₆H₄ | 2,3,4-Cl₃C₆H₂ | —CH₂—CH₂—O—CH₂—CH₂ | | CO | CO | 1 | S | 166-165 |
| 198 | 2-FC₆H₄ | 2,3,4-Cl₃C₆H₂ | H | 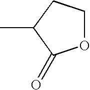 | CO | CO | 1 | S | 269-270 |
| 199 | 2-FC₆H₄ | 2,3,4-Cl₃C₆H₂ | H | 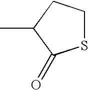 | CO | CO | 1 | S | 220-221 |
| 200 | 2-FC₆H₄ | 2,3,4-Cl₃C₆H₂ | H | C₆H₅ | CO | CO | 1 | S | 220-221 |

### USE EXAMPLES

The herbicidal activity of the compounds of the formula I according to the invention was demonstrated by the following greenhouse experiments:

The cultivation containers used were plastic pots containing loamy sand with approximately 3.0% of humus as the substrate. The seeds of the test plants were sown separately for each species.

For the pre-emergence treatment, the active compounds, which had been suspended or emulsified in water, were applied by means of finely distributing nozzles directly after sowing. The containers were irrigated gently to promote germination and growth and subsequently covered with transparent plastic hoods until the plants had rooted. This cover caused uniform germination of the test plants, unless this was adversely affected by the active compounds.

For the post-emergence treatment, the test plants were first grown to a height of from 3 to 15 cm, depending on the plant habit, and only then treated with the active compounds which had been suspended or emulsified in water. The test plants were for this purpose either sown directly and grown in the same containers, or they were first grown separately as seedlings and transplanted into the test containers a few days prior to treatment. The application rate for the post-emergence treatment was 3 kg of a.s. (active substance)/ha.

Depending on the species, the plants were kept at 10-25° C. or 20-35° C. The test period extended over from 2 to 4 weeks. During this time, the plants were tended, and their response to the individual treatments was evaluated.

The evaluation was carried out using a scale from 0 to 100. 100 means no emergence of the plants, or complete destruction of at least the aerial parts and 0 means no damage, or normal course of growth.

The plants used in the greenhouse experiments were of the following species:

| Bayer code | Common name |
|---|---|
| ABUTH | velvet leaf |
| AVEFA | wild oats |
| CENCY | cornflower |
| CHEAL | lambsquarters |
| | (goosefoot) |
| SETIT | foxtail millet |
| SINAL | white mustard |

At application rates of 3 kg of a.s./ha, the compound of Example 71 (see Table 6) showed very good herbicidal post-emergence action against ABUTH, AVEFA, CENCY, CHEAL, SETIT and SINAL.

At application rates of 3 kg of a.s./ha, the compound of Example 88 (see Table 6) showed very good herbicidal post-emergence action against CENCY, CHEAL and SINAL.

1. A method for controlling undesirable vegetation, which comprises allowing a herbicidally effective amount of at least one compound of the formula I or an agriculturally useful salt thereof to act on plants, their habitat and/or seeds, 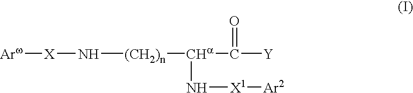
where
X, X¹ independently of one another are —CO— or —SO₂—,
Ar² and Ar^{ω} independently of one another are phenyl, naphthyl, mono- or bicyclic hetaryl having 5 to 10 ring atoms and 1, 2 or 3 hetero atoms, selected from nitrogen, oxygen and sulfur, where phenyl, naphthyl, mono- and bicyclic hetaryl may be unsubstituted or carry one, two, three, four or five substituents selected from the group consisting of halogen, hydroxyl, mercapto, nitro, cyano, CO₂H, HC(O), HC(O)O, C₁-C₁₂-alkyl, C₂-C₁₂-alkenyl, C₂-C₈-alkynyl, C₁-C₁₂-alkoxy, C₂-C₁₂-alkenyloxy, C₃-C₈-alkynyloxy, C₁-C₁₂-alkylthio, C₁-C₁₂-alkylsulfinyl, C₁-C₁₂-alkylsulfonyl, C₁-C₈-alkylcarbonyl, C₁-C₈-alkylcarbonyloxy, C₁-C₈-alkyloxycarbonyl, C₅-C₈-cycloalkyloxycarbonyl, C₁-C₈-haloalkyl, C₁-C₈-haloalkoxy, C₂-C₈-haloalkenyloxy, C₁-C₈-haloalkylthio, C₁-C₈-haloalkylsulfonyl, NH₂, NH—C₁-C₄-alkyl, N(C₁-C₄-alkyl)₂, C₁-C₆-alkoxy-C₁-C₄-alkyl, C₂-C₆-alkenyloxy-C₁-C₄-alkyl, C₃-C₄-alkynyloxy-C₁-C₄-alkyl, C₁-C₆-alkylthio-C₁-C₄-alkyl, C₂-C₆-alkenylthio-C₁-C₄-alkyl, C₁-C₈-alkylcarbonyl-C₁-C₄-alkyl, C₁-C₈-alkylcarbonyloxy-C₁-C₄-alkyl, C₁-C₈-alkyloxycarbonyl-C₁-C₄-alkyl, C₅-C₈-cycloalkyloxycarbonyl-C₁-C₄-alkyl, C₁-C₆-alkoxy-C₁-C₄-alkoxy, C₂-C₆-alkenyloxy-C₁-C₄-alkoxy, C₃-C₄-alkynyloxy-C₁-C₄-alkoxy, C₁-C₆-alkylthio-C₁-C₄-alkoxy, C₂-C₆-alkenylthio-C₁-C₄-alkoxy C₁-C₈-alkylcarbonyl-C₁-C₄-alkoxy, C₁-C₈-alkylcarbonyloxy-C₁-C₄-alkoxy, C₁-C₈-alkyloxycarbonyl-C₁-C₄-alkoxy, C₅-C₈-cycloalkyloxycarbonyl-C₁-C₄-alkoxy, where two substituents located at adjacent carbon atoms of Ar² or Ar^{ω} may also form a C₃-C₅-alkylene chain which may be substituted and in which one or two nonadjacent methylene groups may be replaced by oxygen atoms;
n is 1, 2, 3, 4 or 5 and, 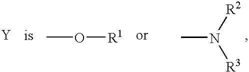
R¹ is hydrogen, C₁-C₁₂-alkyl, C₂-C₁₂-alkenyl, C₃-C₈-alkynyl, C₁-C₁₂-haloalkyl, C₂-C₁₂-haloalkenyl, C₃-C₈-haloalkynyl, where in each case 1, 2 or 3 nonadjacent CH₂ groups may be replaced by oxygen, sulfur or an imino group,
is C₃-C₈-cycloalkyl, C₃-C₈-cycloalkyl-C₁-C₄-alkyl, where the cycloalkyl moieties of the two last-mentioned groups may be partially or fully halogenated, may contain one or two double bonds and/or may carry one, two or three substituents selected from the group consisting of C₁-C₄-alkyl, hydroxyl, C₁-C₄-alkoxy, NH₂, NH-C₁-C₄-alkyl, N(C₁-C₄-alkyl)₂, where in the cycloalkyl moiety 1 or 2 nonadjacent CH₂ groups may be replaced by oxygen, sulfur or an imino group and the cycloalkyl moiety may have one or two carbonyl or thiocarbonyl groups as ring members,
is phenyl-C₁-C₄-alkyl, phenoxy-C₁-C₄-alkyl or hetaryl-C₁-C₄-alkyl having 5 to 10 ring atoms and 1, 2 or 3 hetero atoms, selected from nitrogen, oxygen and sulfur, where the phenyl ring and the hetaryl ring of the three last-mentioned groups may be unsubstituted or carry one, two, three or four substituents selected from the group consisting of halogen, hydroxyl, amino, mercapto, C₁-C₄-alkyl, C₁-C₄-alkoxy, C₁-C₄-alkylcarbonyl, C₁-C₄-alkylcarbonyloxy, C₁-C₄-alkoxycarbonyl, C₁-C₄-haloalkyl, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, cyano and nitro,
R² is hydrogen, C₁-C₃₂-alkyl, C₂-C₁₂-alkenyl, C₃-C₈-alkynyl, C₁-C₁₂-haloalkyl, C₂-C₁₂-haloalkenyl, C₃-C₈-haloalkynyl, where in each case 1, 2 or 3 nonadjacent CH₂ groups may be replaced by oxygen, sulfur or an imino group,
is C₃-C₈-cycloalkyl, C₃-C₈-cycloalkyl-C₁-C₄-alkyl, where the cycloalkyl moieties of the two last-mentioned groups may be partially or fully halogenated, may contain one or two double bonds and/or may carry, one, two or three substituents, selected from the group consisting of C₁-C₄-alkyl, hydroxyl, C₁-C₆-alkoxy, NH₂, NH—C₁-C₄-alkyl, N(C₁-C₄-alkyl)₂, where in the cycloalkyl moiety 1 or 2 nonadjacent CH₂ groups may be replaced by oxygen, sulfur or an imino group and the cycloalkyl moiety may contain one or two carbonyl or thiocarbonyl groups as ring members,
is phenyl, phenyl-C₁-C₄-alkyl, phenoxy-C₁-C₄-alkyl, mono- or bicyclic hetaryl or hetaryl-C₁-C₄-alkyl having in each case 5 to 10 ring atoms and 1, 2 or 3 hetero atoms, selected from nitrogen, oxygen and sulfur, where the phenyl ring and the hetaryl ring of the five last mentioned groups may be unsubstituted or carry one, two, three or four substituents selected from the group consisting of halogen, hydroxyl, amino, mercapto, C₁₋C₄-alkyl, C₁-C₄-alkoxy, C₁-C₄-alkylcarbonyl, C₁-C₄-alkylcarbonyloxy, C₁-C₄-alkoxycarbonyl, C₁-C₄-haloalkyl, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, cyano and nitro,
R³ has one of the meanings given for R² or together with R² and the nitrogen atom to which they are attached forms a saturated or unsaturated nitrogen heterocycle having 5, 6 or 7 ring atoms which may contain 1 or 2 additional heteroatoms, selected from nitrogen, oxygen and sulfur, and/or 1 or 2 carbonyl or thiocarbonyl groups as ring members and which may be unsubstituted or carry one, two, three or four substituents.

2. The method as claimed in claim 1, where Ar² and Ar^{ω} independently of one another are phenyl, thienyl, furanyl, pyrrolyl, pyridyl, pyrimidinyl, naphthyl or quinolinyl which may be substituted or carry 1, 2 or 3 substituents selected from the group consisting of halogen, hydroxyl, mercapto, nitro, cyano, CO₂H, HC(O), HC(O)O, C₁-C₈-alkyl, C₂-C₈-alkenyl, C₂-C₈-alkynyl, C₁-C₈-alkoxy, C₂-C₈-alkenyloxy, C₃-C₈-alkynyloxy, C₁-C₈-alkylthio, C₁-C₈-alkylsulfinyl, C₁-C₈-alkylsulfonyl, C₁-C₈-alkylcarbonyl, C₁-C₈-alkylcarbonyloxy, C₁-C₈-alkyloxycarbonyl, C₅-C₈-cycloalkyloxycarbonyl, C₁-C₈-haloalkyl, C₁-C₈-haloalkoxy, C₂-C₈-haloalkenyloxy, C₁-C₈-haloalkylthio, C₁-C₈-haloalkylsulfonyl, NH₂, NH—C₁-C₄-alkyl, N(C₁-C₄-alkyl)₂, where two substituents located at adjacent carbon atoms may also form a C₃-C₅-alkylene chain in which one or two nonadjacent groups may be replaced by oxygen atoms.

3. The method in claim 1 where Y is a group R¹O,
where R¹ is selected from the group consisting of hydrogen, C₁-C₁₂-alkyl, C₂-C₁₂-alkenyl, C₃-C₈-alkynyl, hydroxyl-C₁-C₄-alkyl, amino-C₁-C₄-alkyl, C₁-C₆-alkoxy-C₁-C₄-alkyl, C₁-C₆-alkoxy-C₁-C₄-alkoxy-C₁-C₄-alkyl, C₁-C₆-alkylamino-C₁-C₄-alkyl, di-(C₁-C₆-alkyl)amino-C₁-C₄-alkyl, C₂-C₆-alkenyloxy-C₁-C₄-alkyl, C₃-C₄-alkynyloxy-C₁-C₄-alkyl, C₁-C₆-alkylthio-C₁-C₄-alkyl, C₂-C₆-alkenylthio-C₁-C₄-alkyl, where the 8 groups mentioned above may also carry 1, 2, 3, 4 or 5 halogen atoms, selected from the group consisting of fluorine and chlorine,
is C₃-C₈-cycloalkyl, C₃-C₈-cycloalkyl-C₁-C₄-alkyl or C₅-C₆-cycloalkenyl, where the cycles of the three last mentioned groups may, instead of a methylene group, contain an oxygen or sulfur atom, may carry a carbonyl group and may be unsubstituted or carry one to four substituents, selected from the group consisting of fluorine, chlorine, C₁-C₄-alkyl and C₁-C₄-alkoxy,
is phenyl-C₁-C₄-alkyl, 2-, 3- or 4-pyridyl-C₁-C₄-alkyl, 2- or 3-thienyl-C₁-C₄-alkyl, which may be unsubstituted or carry 1, 2, 3 or 4 substituents selected from the group consisting of halogen, C₁-C₄-alkyl, C₁-C₄-alkoxy, C₁-C₄-haloalkyl, C₁-C₄-haloalkoxy, cyano and nitro.

4. The method as claimed in claim 1, where Y is a group 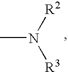
where
R² is selected from the group consisting of hydrogen, C₁-C₁₂-alkyl, C₂-C₁₂-alkenyl, C₃-C₈-alkynyl, hydroxy-C₁-C₄-alkyl, amino-C₁-C₄-alkyl, C₁-C₆-alkoxy-C₁-C₄-alkyl, C₁-C₆-alkoxy-C₁-C₄-alkoxy-C₁-C₄-alkyl, C₁-C₆-alkylamino-C₁-C₄-alkyl, di-(C₁-C₆-alkyl)amino-C₁-C₄-alkyl, C₂-C₆-alkenyloxy-C₁-C₄-alkyl, C₃-C₄-alkynyloxy-C₁-C₄-alkyl, C₁-C₆-alkylthio-C₁-C₄-alkyl, C₂-C₆-alkenylthio-C₂-C₄-alkyl, where the 8 abovementioned groups may also carry 1, 2, 3, 4 or 5 halogen atoms, selected from the group consisting of fluorine and chlorine,
is C₃-C₈-cycloalkyl, C₃-C₈-cycloalkyl-C₁-C₄-alkyl or C₅-C₆-cycloalkenyl, where the cycles of the three last mentioned groups may, instead of a methylene group, contain an oxygen atom, sulfur atom or an NH group, may contain a carbonyl group and may be unsubstituted or carry one to four substituents, selected from the group consisting of fluorine, chlorine, C₁-C₄-alkyl and C₁-C₄-alkoxy,
is phenyl, 2- or 3-thienyl, 2-, 3- or 4-pyridyl, 2-, 4- or 5-thiazolyl, 2-, 4- or 5-pyrimidinyl, 3- or 4-pyridazinyl, 2-benzothiazolyl, phenyl-C₁-C₄-alkyl, phenoxy-C₁-C₄-alkyl, 2-, 3- or 4-pyridyl-C₁-C₄-alkyl, 2- or 3-thienyl-C₁-C₄-alkyl, where the abovementioned aromatic or heteroaromatic groups may be unsubstituted or carry 1, 2, 3 or 4 substituents selected from the group consisting of halogen, C₁-C₄-alkyl, C₁-C₄-alkoxy, C₁-C₄-haloalkyl, C₁-C₄-haloalkoxy, cyano and nitro,
R³ is hydrogen or C₁-C₄-alkyl or together with R² and the nitrogen atom to which they are attached forms a saturated 5-, 6- or 7-membered nitrogen heterocycle which may be unsubstituted or carry 1, 2, 3 or 4 substituents selected from the group consisting of C₁-C₄-alkyl and C₁-C₄-alkoxy and in which one methylene group may be replaced by an oxygen atom, a sulfur atom, an NH or a C₁-C₄-alkylimino group.

5. The method as claimed in claim 1, where the compounds of the formula I have the S configuration at the α-carbon atom.

6. The method as claimed in claim 1, where X and X¹ in the formula I are C═O. are C═O.

7. (canceled)

8. A 2,ω-diaminocarboxylic acid compound of the formula I as setforth in claim 1 and their agriculturally compatible salts, wherein Y is a group 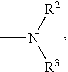
where
R² is C₁-C₁₂-alkyl, C₂-C₁₂-alkenyl, C₃-C₈-alkynyl, C₁-C₁₂-haloalkyl, C₂-C₁₂-haloalkenyl, C₃-C₈-haloalkynyl, where in each case 1, 2 or 3 nonadjacent CH₂ groups may be replaced by oxygen, sulfur or an imino group,
is C₃-C₈-cycloalkyl, C₃-C₈-cycloalkyl-C₁-C₄-alkyl, where the cycloalkyl moieties of the two last-mentioned groups may be partially or fully halogenated, may contain one or two double bonds and/or may carry, one, two or three substituents, selected from the group consisting of C₁-C₄-alkyl,hydroxyl, C₁-C₄-alkoxy, NH₂, NH—C₁-C₄-alkyl, N(C₁-C₄-alkyl)₂, where in the cycloalkyl moiety 1 or 2 nonadjacent CH₂ groups may be replaced by oxygen, sulfur or an imino group and the cycloalkyl moiety may contain one or two carbonyl or thiocarbonyl groups as ring members,
is phenyl, phenyl-C₁-C₄-alkyl, phenoxy-C₁-C₄-alkyl, mono- or bicyclic hetaryl or hetaryl-C₁-C₄-alkyl having in each case 5 to 10 ring atoms and 1, 2 or 3 hetero atoms, selected from nitrogen, oxygen and sulfur, where the phenyl ring and the hetaryl ring of the five last mentioned groups may be unsubstituted or carry one, two, three or four substituents selected from the group consisting of halogen, hydroxyl, amino, mercapto, C₁-C₄-alkyl, C₁-C₄-alkoxy, C₁₋C₄-alkylcarbonyl, C₁-C₄-alkylcarbonyloxy, C₁-C₄-alkoxycarbonyl, C₁-C₄-haloalkyl, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, cyano and nitro,
R³ is hydrogen or has one of the meanings given for R² or together with R² and the nitrogen atom to which they are attached forms a saturated or unsaturated nitrogen heterocycle having 5, 6 or 7 ring atoms which may contain 1 or 2 additional heteroatoms, selected from nitrogen, oxygen and sulfur, and/or 1 or 2 carbonyl or thiocarbonyl groups as ring members and which may be unsubstituted or carry one, two, three or four substituents.

9. A process for preparing 2,ω-diaminocarboxylic acid compounds of the formula I, as claimed in claim 8, which comprises reacting a compound of the formula II 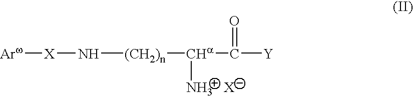
where Ar^{ω}, X, n are as defined above, Y is as defined in claim 8 and X^{⊖} is a monovalent anion or an anion equivalent of a mineral acid with an aryl halide of the formula III
Ar²-X¹-Hal (III)
where Ar² and X¹ are as defined above and Hal is chlorine, bromine or iodine.

10. The process as claimed in claim 9, wherein initinally compound of the formula II is prepared by reacting a partially protected 2,ω-diaminocarboxylic acid of the formula IV or an acid addition salt thereof. 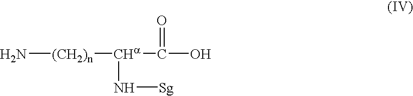
in which n is as defined above and Sg is a protective group in a first step with an acyl halide of the formula V
Ar^{ω}-X-Hal (V)
where Ar^{ω} and X are as defined above and Hal is chlorine, bromine or iodine, the resulting compound of the formula VI 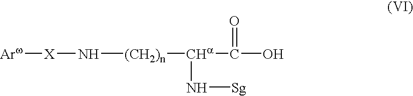
is reacted with an amine of the formula R²R³NH, where R² and R³ are as defined above, in the presence of a suitable condensing agent, and the protective group Sg is removed, giving a compound of the formula II.

11. A composition, comprising at least one 2,ω-diaminocarboxylic acid compound of the formula I or an agriculturally useful salt of I as claimed in claim 8 and customary auxiliaries.

12. A compound of the formula II 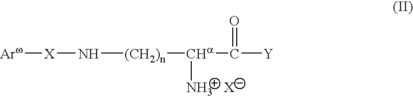
in which Ar^{ω}, X, n and X^{⊖} are as defined in claim 9.
